(19) United States
(12) Patent Application Publication
(10) Pub. No.: US 2015/0338477 A1
(43) Pub. Date: Nov. 26, 2015

(54) AN ACTIVE TRACKING SYSTEM AND METHOD FOR MRI

(71) Applicants: Ehud J. SCHMIDT, (US); Wei WANG, (US); Tina KAPUR, (US); Charles L. DUMOULIN, Cincinnati, OH (US); Robert A. CORMACK, (US); Akila VISWANATHAN, (US); THE BRIGHAM AND WOMEN'S HOSPITAL, Boston, MA (US); CHILDREN'S HOSPITAL MEDICAL CENTER, Cincinnati, OH (US)

(72) Inventors: Ehud J. Schmidt, Newton, MA (US); Wei Wang, Auburndale, MA (US); Tina Kapur, Cambridge, MA (US); Charles L. Dumoulin, Cincinnati, OH (US); Robert A. Cormack, Milton, MA (US); Akila Viswanathan, Cambridge, MA (US)

(21) Appl. No.: 14/759,900

(22) PCT Filed: Jan. 8, 2014
(86) PCT No.: PCT/US2014/010656

§ 371 Date: Jul. 8, 2015

Related U.S. Application Data
(60) Provisional application No. 61/750,473, filed on Jan. 9, 2013.
(60) Provisional application No. 61/779,037, filed on Mar. 13, 2013.
(60) Provisional application No. 61/847,321, filed on Jul. 17, 2013.
(60) Provisional application No. 61/875,367, filed on Sep. 9, 2013.

(51) Int. Cl. G01R 33/28; G01R 33/56; G01R 33/565; A61B 17/34

(52) U.S. Cl. CPC G01R 33/286; A61B 17/3403; G01R 33/5608; G01R 33/56563; A61B 2562/12

(57) ABSTRACT

A composite system for use in conjunction with an MR-imaging procedure. One composite system includes a fully-metallic filament, such as a needle or guide-wire, equipped with flat MR RF-receiver microcoil disposed such that a normal to the coil's plane is substantially transverse to the filament's axis. The microcoil is electrically connected to external device to register change of position and orientation of the tip during the navigation of the filament. Alternative composite system includes a filament made from different materials. The very tip includes diamagnetic and non-metallic tube tightly fit around geometrically-modified portion of the main body and carries at least one microcoil electrically connected to external device to register change of position and orientation of the tip during the filament navigation. Data representing co-registration of the position and/or orientation of filament is fed back to the system to improve navigation accuracy and precision.

### CROSS-REFERENCE TO RELATED APPLICATIONS

The present application relates to and claims priority from each and every one of U.S. provisional applications Nos. 61/750,473 (attorney docket no. 129319.00165) filed on Jan. 9, 2013 and titled “Metallic Needle Insertion and Tracking systems for MRI”; 61/779,037 (attorney docket no. 129319.00168) filed on Mar. 13, 2013 and titled “Tracking System for MRI”; 61/847,321 (attorney docket no. 129319.00192) filed on Jul. 17, 2013 and titled “Active MRI-based positional tracking of metallic objects”; and 61/875,367 (attorney docket no. 129319.00193) filed on Sep. 9, 2013 and titled “Active MRI-based positional tracking of metallic objects”.

The disclosure of each of the above-identified patent applications is incorporated herein by reference in its entirety

### TECHNICAL FIELD

The present invention relates to magnetic-resonance-based interventional procedures and systems and, in particular, to an interventional needle device, a position of which is subject to tracking during an MRI-guided surgical or interventional procedure.

### BACKGROUND

Metallic needles are commonly used in interventional applications due to their mechanical strength of resistance (characterized by a high Young's modulus and a low, less than 0.3, Poison's ratio) to compressive and bending forces. The metallic needles also exhibit a high degree of elasticity (i.e., possess a high yield strength) and are non-brittle (i.e., possess a high ultimate tensile strength). Examples of interventional applications include tumor (or other pathology) biopsy procedures; thermal-ablative (using radio-frequency, RF, or microwave sources) or cryo-ablative (using a cooling mechanism such as the Joule-Thompson effect, for example) procedures where a metallic needle, such as a cannula, is used to transport the ablation delivery device to the proximity of the targeted pathology; radiation treatment of pathologies, where the needles are used to bring radioactive seeds (or mechanically-soft enclosures, referred to as “catheters” in the Radiation Oncology nomenclature, into which the radioactive seeds can later be inserted) to the proximity of the targeted pathology; chemical ablative or therapeutic procedures, where therapeutic chemical or biological agents are delivered to the tumor; as well as neurovascular and cardiovascular interventions, in which catheters utilize metallic braids to enable remote (at a separation on the order of 1.5 meters) deflection and rotation during navigation within the vascular anatomy.

An MR-imaging (MRI) modality is often employed to detect the pathology because of the enhanced contrast-to-noise ratio (CNR) between soft tissues afforded by the MRI system, which can be used to enable improved navigation to the target and improved differentiation between the pathology and its surroundings. As a result, it may be beneficial to perform the interventional procedure while the patient is inside the bore of the MRI system. Advantages of performing the intervention inside the MRI system are particularly pronounced when the pathology is present within soft tissue, which can deform non-rigidly and non-uniformly (for example, to different degrees in different directions). Indeed, performing the intervention procedure on such deformable tissues outside the MRI system, based on the information obtained with the MRI system, is not straightforward, as the patient tends to move between the MR-imaging session and the intervention.

During the magnetic-resonance (MR) based interventional procedures, the position and orientation of a catheter or needle can be rather precisely tracked using MR-based tracking methods, such as active tracking using one-dimensional MRI profiles (MR Tracking), or tracking utilizing micro-coils which detect electrical fields induced by temporal changes in the magnetic-gradient fields. The presence of metal, such as found in the metallic needle, within the field of view of the MR tracking element affects the tracking quality by causing in-homogeneities in both the static and radio-frequency (RF) magnetic fields around the needles, a result of the strong paramagnetic and electrical conductivity properties of the metallic needle. Moreover, metallic needles that are long (for example, about 15 cm) act as radiofrequency (RF) antennae that operably couple to the tracking coils, thereby distorting the appearance of the tracking signals, so that spatial localization is complicated. As a result, tracking of metallic devices is presently limited to tracking of non-metallic objects (referred to as “hand-holders” or “device-holders”) that are attached to the proximal ends (the side which, in operation, is usually outside of the body) of the needles; the “device-holders”, however, cannot detect the bending of the needle shaft which occurs during insertion of the needle into human tissue and, as a result, they cannot facilitate accurate determination of the position of the needle's tip.

Needles made from non-ferromagnetic metals (i.e. from metals that are paramagnetic, but not ferromagnetic) can be tracked passively (based, for example, on the magnetic susceptibility of the metals, that is higher than that of the surrounding soft tissue). The disadvantage of this approach, however, is that high-resolution MRI sequences responsive to differences in magnetic susceptibility must be used, which requires far longer imaging times than those of active tracking (for example, about 60 seconds as compared to about 0.025 to 0.04 second to achieve comparable spatial accuracy). In addition, the accuracy of the needle's tip localization is lower (about 3 mm in passive tracking, as compared to about 0.5 mm in active tracking). When multiple needles are used, tracking of the individual paths of the individual needles with a passive approach becomes problematic, especially if different needles end up in proximity to one another or have their paths crossed. Passive tracking has an additional shortcoming in that the lengthy time required to accomplish the tracking procedure is not conducive to carrying out rapid (on the order of several seconds) MRI imaging of the immediate neighborhood of the needle's tip during navigation, using the instantaneous coordinates and orientation of the tip, a procedure which is possible to perform with the use of active tracking, since updates to this position are obtained several times per second. Yet another shortcoming of passive tracking is the inability to perform motion-compensated imaging. High-spatial-resolution MRI images typically require several minutes to acquire, which encompasses multiple cycles of physiological motion (human cardiac motion, typically at 1-2 cycles per second, includes non-rigid motion over about 1 to 2 cm, while respiratory motion, typically at 0.2-0.5 cycles/sec, includes non-rigid motion over about 2 to 3 cm). Since the duration of an MRI acquisition is longer than the physiological cycle, the anatomy is continuously moving while it is being imaging, which results in blurred (lower resolution) and even artifactual images. If a needle or catheter, equipped with active tracking micro-coils, is placed inside the anatomy, then this device can sense the instantaneous direction and magnitude of the motion. As a result, MRI imaging sequences can be constructed that interleave between active tracking and imaging segments, whereby the imaging segments are fed with the detected changes in position and shifted appropriately, so that the imaging sequence is performed in a static (non-moving) frame of reference relative to the anatomy, which results in MRI images with substantially reduced image blurring or image artifacts. Imaging of moving anatomy is commonly performed by utilizing sensors placed on the body surface or image-based navigators, but these methods cannot accurately correct for motion in cases where the anatomic motion is not of a rigid-body nature (i.e. it varies greatly with position), so they tend to over- or under-compensate for such motion, and they also can result in very lengthy scans. In such cases of physiological motion, the correction provided by the microcoils, since they are positioned far closer to the target tissues, provides a far better approximation of the magnitude and direction of motion at the target tissue, and thereby in images with better removal of motion artifacts.

Yet another approach to tracking of interventional needles using MRI methods is to replace the metallic needles entirely with a hard non-ferrous substance (such as a ceramic or a composite material, for example: fiber glass or carbon fiber). However, a lengthy needle of a small diameter (for example, at least 8 cm long by less than 2 mm thick), made of most of the composites or ceramic materials, is inflexible and exceeds its elastic limit when bent at an angle of a few degrees. Accordingly, such a needle is likely to break when used to push aside or cut-through soft tissue, which is unacceptable. Moreover, machining such needles is complicated, thereby limiting easily producible needle shapes. Finally, observing the entire length of the needle at once with an active tracking method is problematic, and an optimized approach may therefore require the detection of the needle tip actively, while the rest of the needle is passively detected.

It is appreciated, therefore, that there remains a need for a needle system and method for MR-based tacking of such a needle that overcome shortcomings of the related art and, in operation, facilitate the creation of conditions that help avoid distortions of the image provided by the MRI system on the one hand and yet are able to position the needle accurately with respect to a desired location in the tissue.

### SUMMARY

Embodiments of the invention provide a method for making a needle for an interventional device. Such method includes attaching a tubular distal needle segment, made of a non-metallic and either diamagnetic or paramagnetic material, to a distal end of the carrying portion of the needle made of metal. The ratio of a first value representing a length of the carrying portion and a second value representing a length of the tubular distal needle segment is at least 10. The method additionally includes adding a coil made of electrically conductive wire around the tubular distal needle segment; and providing an electrical output to the coil. In a specific implementation, the distal end of the carrying portion is dimensioned to ensure friction fit of the distal end inside the tubular needle element. The method further includes providing an electrically conducting member between the coil and the electrical output; and encasing at least the carrying portion of the needle in a plastic tubing to pass the electrically conducting member inside the tubing. Embodiments of the invention additionally provide a needle formed according to the above-identified method, and a system and method for MR-guided tracking of the needle guidance.

One implementation of the invention provides a system adapted for use with a system for actively tracking of a position of a device within a magnetic resonance imaging (MRI) scanner. The system of the invention includes a fully-metallic filament extended along an axis and including proximal and distal ends, a first length, and extended along an axis, such filament having a substantially flat surface along the first length. The system additionally includes at least one MR receiver coil including at least one first loop that forms a first electrically-conductive trace disposed in a first plane parallel to the substantially flat surface such that a normal to the plane is transverse to the substantially flat surface. At least one coil has electrical terminals electrically extended towards the proximal end. In one embodiment, the at least one coil further includes at least one second loop that forms a second electrically-conductive trace disposed in a second plane parallel to the substantially flat surface and electrically connected to the at least one first loop, such as to define a length of the at least one coil as a sum of lengths of the first and second electrically-conductive traces, the first and second planes being different and parallel to one another. The needle may further contain a plastic sheath encasing at least the fully-metallic filament, wherein the first plastic portion and the plastic sheath are dimensioned to form a gap there between, and wherein an electrical extension of a terminal toward the proximal end is disposed in the gap.

### BRIEF DESCRIPTION OF THE DRAWINGS

The invention will be more fully understood by referring to the following Detailed Description in conjunction with the Drawings, of which:

FIGS. 1 and 2 are plan side views of different portions of an embodiment of the invention;

FIGS. 3A and 3B are diagrams providing examples of the geometric formatting of a printed circuit which embodies two layers of a MR radio-frequency (RF) receiver coil placed at the distal end of the metallic portion of the embodiment of FIG. 1;

FIG. 4 is a cross-sectional MRI image of the embodiment of FIG. 1 disposed inside a catheter;

FIG. 5A is a graph related to the recording of an MR-tracking;

FIG. 5B is a graph representing recording of an MR-tracking of an embodiment of the invention;

FIGS. 6A, 6B, 6C are plots representing a recording of an MR-tracking of FIG. 5B with signal averaging over 1, 2, and 4 readings, respectively, to reduce noise and detect the true peak position;

FIG. 7 is a flow-chart illustrating schematically an embodiment of a false peak removal algorithm of the invention;

FIGS. 8A, 8B are plots of real and imaginary parts of a noisy MR-tracking signal (recorded at low levels of signal to noise ratio) and, overlapped with such parts, the corresponding portions of the signal after the false-peaks have been removed with the use of an algorithm of the invention;

FIG. 8C is a plot illustrating an absolute value of the signal corresponding to the plots of FIGS. 8A, 8B in the time domain;

FIGS. 9A, 9B illustrate in frequency domain, respectively, a recorded signal representing active MRI-tracking of the metallic object equipped with a flat coil according to the embodiment of the invention before and after the false-peak removal filtering algorithm has been applied to the results of tracking;

FIG. 10A is a plot illustrating results of active MRI-tracking of an embodiment of the invention averaged according to approach of related art, which results in no obvious determination of an MR peak;

FIG. 10B is a plot illustrating improvement of the approach of the related in comparison with that of FIG. 10B, when the false-peak removal method, according to an embodiment of the invention, makes a difference in distinguishing and identification of an MR-peak;

FIGS. 11A and 11B schematically illustrate a metallic needle equipped with the MR RF coil according to an embodiment of the invention and the ambient medium defined to carry the simulation of the radio-frequency (RF) magnetic field distribution around the needle while inside the MRI-system;

FIGS. 12A, 12B show the orientation of sagittal, coronal, and axial planes in reference to the geometry of an embodiment of the invention fabricated with the use of tungsten;

FIGS. 13A, 13B, 13C illustrate the spatial distribution of the RF magnetic field in sagittal, coronal, and axial planes calculated under stated conditions;

FIGS. 14A, 14B provide, for comparison, distribution of magnetic field corresponding to related embodiments in which the needle is made of different materials;

FIG. 15 is a diagram illustrating a metallic needle of the invention having three slots for attaching the flat RF coils;

FIGS. 16 and 17 are plan side views of different portions of an embodiment of the invention;

FIGS. 18A and 18B are diagrams providing examples of geometric formatting of the distal end of the metallic portion of the embodiment of FIG. 16;

FIG. 19 is a cross-sectional view of the embodiment of FIG. 16 disposed inside a catheter;

FIG. 20A is a general perspective view of the embodiment of FIG. 16;

FIG. 20B is a graph representing recording of an MR-tracking of an embodiment of the invention;

FIG. 20C combines a depiction of the distal end of the embodiment of FIG. 20A and an MR image that identifies the two microcoils adjacent to the distal end;

FIG. 21A is a diagram illustrating a composition of a system of the invention;

FIG. 21B is an example of a single-channel tracking receiver for use with the embodiment of FIG. 21A;

FIGS. 22A, 22B, 22C, 22D, and 22E depict a MR tracking sequence diagram. The pulse sequence is used to multiplex the acquisition of the coil position information and to provide a description of the coil's 3D coordinates with four pulse excitations.

FIG. 23 is an illustration of the eight-receiver tracking module built according to an embodiment of the invention.

### DETAILED DESCRIPTION

Below, a discussion is presented of a device and an approach for using the device by which the “MRI-tracking” method can be implemented to track position(s) and orientation(s) of metallic objects while inside an MRI scanner. The problem of improving the navigation of the interventional device to the target and high-quality differentiation between the pathology and its surroundings with the use of the interventional device is solved by creating a composite interventional device that substantially maintains the mechanical properties of the conventional metallic needle while obviating the tracking and imaging artifacts caused by noise and interference attributed to the conventional needle being fully metallic.

The idea of the present invention stems from the realization that an interventional needle that constitutes a judiciously designed composite system (whether passive or active, in which case a portion of such composite system is structured to take part in the tracking process by generating an associated wave registrable with an appropriate detector, as opposed to a simple metallic needle) can substantially aid the process of not only tracking the needle-based composite system but also the process of repositioning/relocation of such system within the tissue.

For example, structuring an active composite system by juxtaposing a metallic object (such as a metallic needle) to be MRI-tracked with a radio-frequency (RF) coil the geometry of which has an RF radiation (“lobe”) pattern tunable to project this lobe away from the surface of the metallic object, causes the tracking MRI-signal to approach, in practice, the MRI-signal that is undistorted by the presence of the metallic body in the MRI scanner and that, otherwise, can only be obtained in the far-field with respect to the surface of the metallic object.

As another example, structuring a passive composite system as a metallic/non-metallic composite needle facilitates the MR-guided interventional procedure such as, for example, radiation brachytherapy treatment of cervical and prostate tumors because the non-metallic portion of the needle does not interfere with MR imaging. For example, a significant portion of the base of needle, close to the handle, may include traditional metallic materials that provide strength and stifthess to the passive composite needle system, while a distal tip includes non-metallic composite materials that do not compromise the mechanical properties of the needle. About the tip of the composite needle there may be disposed actively-tracked MRI receivers (such as coils, for example), which substantially aid in the tracking of the needle and its rotation.

As already alluded to above, active tracking a position of a metallic object (with emphasis on surgical or interventional devices, for example), is difficult when such active tracking is carried out with the use of Electromagnetic (EM) tracking methods (such as magnetic-field-based or electrical-voltage-based) or MRI-based tracking methods (such as, for example, “MR-tracking”, that is an MRI Radio Frequency-projection based method; or “Robin medical” , an MRI-gradient magnetic-induction based method). One of the problems encountered during such tracking is caused by the tendency of a metallic object to distort, in its proximity, the magnetic and electric fields. As the linearity of these fields is relied upon for determining the object's position(s), these positions are likely to be wrongly calculated. As a result, practical implementation of the tracking of a metallic device in the interventional setting requires the use of X-ray (ionizing) radiation.

Alternatively, or in addition, a portion of the (or the entire) metallic device is being substituted in practice with a portion made of a non-metallic material. Such substitutions, however, may cause problems in operation of the interventional device. For example, while proceedings of the Congenital and Structural Interventions Society's annual conference (Jun. 21-15, 2011) reported the use of a guide-wire (suited for clinical trials and MRI-guided interventions) with the shaft constructed of glass-fiber material, the lowered mechanical properties of such guide-wire were acknowledged to be inadequate for safety purposes, causing breakage while inside the tissue, and the need for MRI compatible equipment, especially guide-wires and catheters, was emphasized. (See also “Magnetic resonance-guided cardiac interventions with the use of magnetic resonance-compatible devices” by Tzifa et al., available at http://circinterventions.ahajournals.org/content/3/6/585.full, the contents of which are incorporated herein by reference). It is recognized, therefore, that translation of MR-guided interventions into subjects has been limited by the lack of MR-compatible and safe non-metallic equipment such as MR guide-wires with mechanical characteristics similar to standard guide-wires.

Another method for active-tracking is the use of an attachment (an external piece referred to as a device handle) to the devices. Each of the alternative and practically-used, at the moment, approaches enables positioning of the tracking sensor(s) at some distance from the metallic portion of the device, as a result of which the interference of the metallic portion of the device with the field of interest can be reduced. The “device handle” approach, however, is only practically suitable for rigid devices, since the bending of a non-rigid object at a point away from the handle cannot be known.

The use of so-called “passive tracking” of the metallic devices, which utilizes image artifacts that a metallic object creates to determine its position, is practically possible but such passive tracking is less accurate spatially, has a lower temporal resolution, and requires the exclusive use of the scanner for the localization of the object's position. Practice shows that passive tracking is also highly dependent on the method of imaging (and, in particular, on imaging sequence, spatial resolution, as well as shape and orientation of the object). Another shortcoming of the passive tracking methodology is the inability to practically implement imaging which is guided with respect to the instantaneous position of the device (“Guided Imaging ”, i.e. imaging at the tip of the device, which follows the position of the tip as it is displaced), which is a result of the fact that locating the position and orientation of the device tip requires an extensive amount of time, relative to the time in which the device remains at a single location, and that the accuracy of this localization is insufficient for purposes of accurately defining the imaging planes. Guided Imaging is known to aid the clinician in revising the insertion path of the device and can reduce the possibility of unwanted perforation and/or invasion of a critical biological structure, so that this deficiency is important clinically.

### Examples of Single-Material Composite Implementations.

In one implementation, the device of invention addresses the unsolved-to-date need for a structurally-reliable, strong, stiff, sharp and otherwise medically preferred, fully-metallic interventional catheterization device (such as a catheter, guide-wire, or needle) that does not impede the ability of MRI-related measurement modalities to actively track the positiion of the device. Accordingly, the embodiment includes a fully-metallic catheter, needle, or quidewire equipped with an auxiliary MR RF receiver coil judiciously structured to enable accurate and quick tracking of the fuly-metallic catheter needle, or quide-wire inside the MRI system. This includes non-rigid devices, where tracking using an external attached “handle” was shown to be insufficiently accurate. The fact that the user of an MRI system equipped with an embodiment of the invention does not have to employ an exploratory tool such as, for example, a brachytherapy needle that is only partially made of metal, but can utilize a fully-metallic needle that is structurally and, in addition, by being actively tracked, operationally superior to any other alternative used today, secures operational advantages not realized by the related art up to date: unique (mechanical, elastic, thermal, etc.) properties of a metallic interventional device are preserved and optimized for performance of specific imaging tasks. Among such tasks there is a task of precise and accurate localization of devices placed around, or within, a patient during MRI diagnostic imaging or MRI-guided interventions (sensors, probes, guidewires, sheathes, catheters, needles, etc.). Finally, the fact that the embodiments of the invention make it possible to utilize previously rejected and/or viewed as operationally inappropriate fully-metallic interventional devices during the MRI procedure, makes the measurements of displacement of such objects (and its derivatives) as well as the related measurement of the surrounding anatomy possible.

While the examples of the embodiment disclosed below discuss embodiments of a interventional needle, it is appreciated that other interventional devices such as, for example, guide-wires and catheters structured in a fashion similar to that disclosed in this application have also been considered and are within the scope of the present invention.

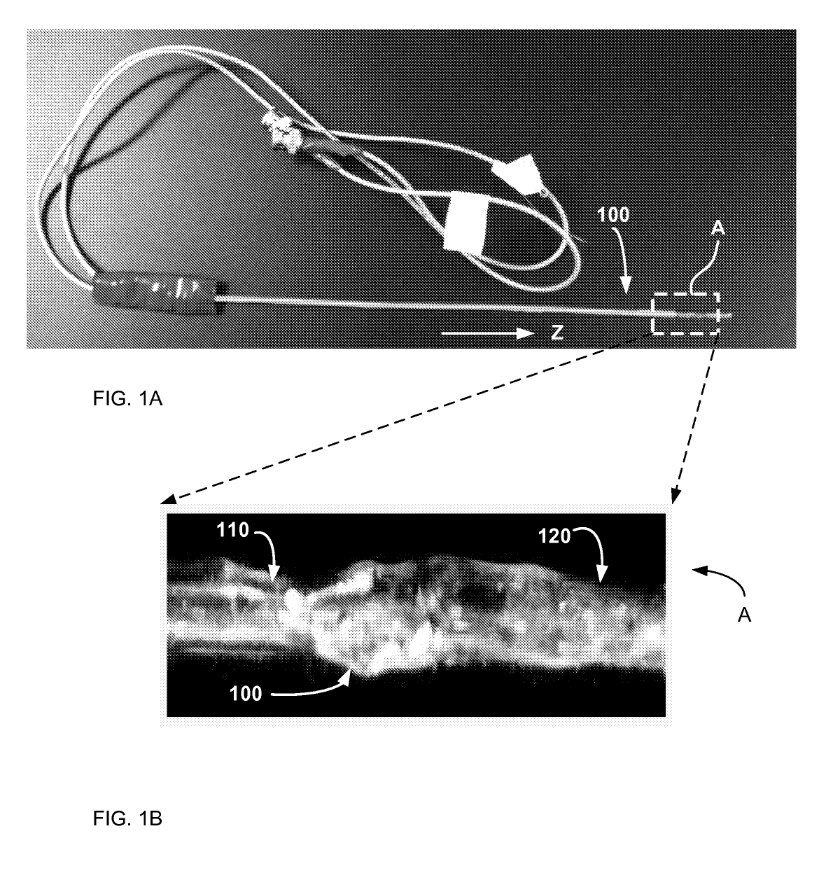

Referring to FIGS. 1A and 1B, a commercially-available MRI-compatible (tungsten-based) Radiation Oncology cervical-cancer Brachytherapy needle 100 was modified by adding to such needle two rectangular-shaped MR receiver micro-coils 110, 120 in such orientation so that the corresponding RF lobe pattern of each of the coils 110, 120 was orthogonal to the a surface of the metallic needle 100 (i.e., in reference to FIG. 1A, orthogonal to the z-axis) and, therefore, to the shaft of the needle. FIG. 1B is an enlarged photograph illustrating a portion A of the needle 100 carrying the coils 110, 120.

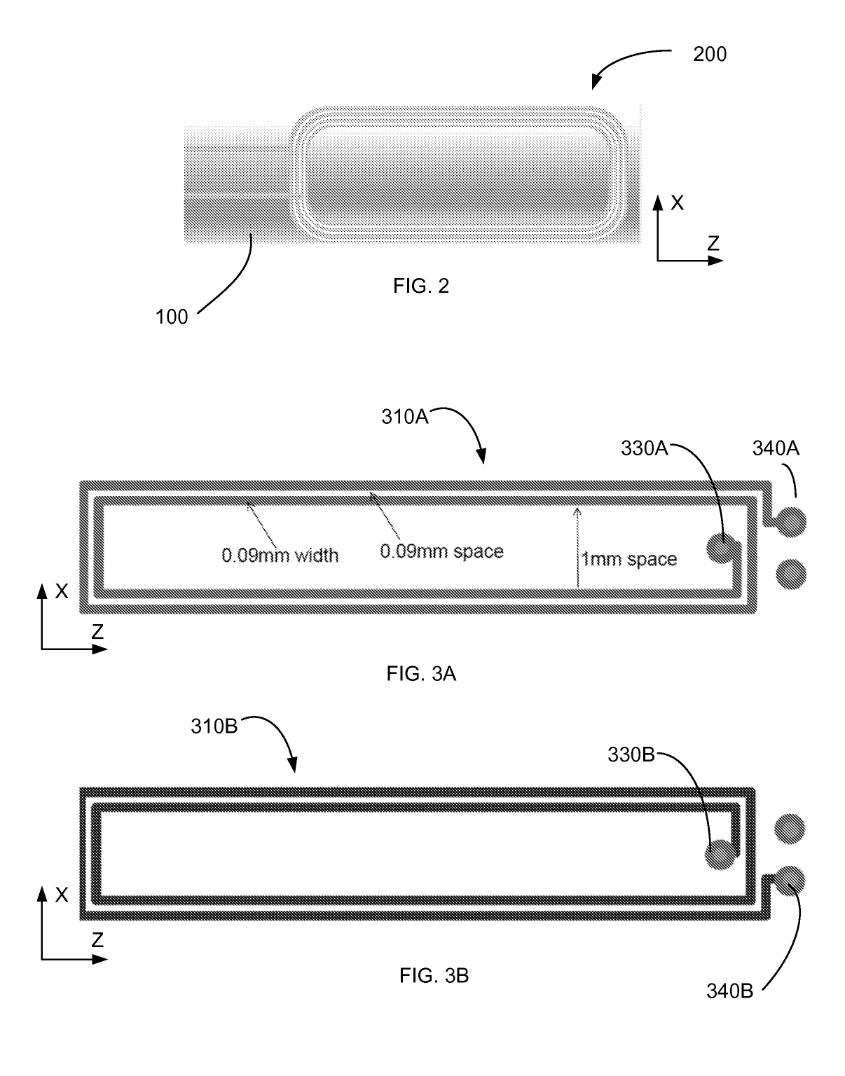

An example 200 of a substantially flat, thin, and containing four metallic loops RF receiver coil (such as any of the coils 110, 120) is shown in FIG. 2. In this implementation, the dimensions of the coil 200 were about 1.2 mm in width by 7 mm in length, and the separation from the surface of the needle 100, defined by the thickness of the flexible integrated circuit on which the loop 200 as integrated, was about 0.1 mm. FIGS. 3A, 3B show two layers (the top layer 310A and the bottom layer 310B) of a related two-layer embodiment of the approximately 8 mm long RF receiver coil 320, with geometrical dimensions of the loops indicated in FIG. 3A. The electrical terminals of the layers 310A, 310B are denoted, respectively, as 330A, 330B, 340A, 340B.

While an embodiment of the substantially flat coil can be generally structured as a conventional, structurally continuous multi-loop spiral (the multiple inner loops of which encircle progressively smaller areas, for example, and in which the multiple loops are defined in the same plane), according to a specific embodiment of the invention the example of which is shown in FIG. 2, the multiple loops of the coil are separated into groups. The groups of coils—as shown, the groups defining the layers 310A, 310B of the coil—are operably cooperated with one another by disposing the layers on top of one another and electrically connecting the terminals 330A, 330B. As a result, the overall two-layer RF-coil structure 200, while containing four loops, has only two loops in each layer and, therefore, a bigger “clear aperture” (the area encircled by the loops) than a four-loop flat coil in which the four loops are conventionally coordinated in a continuous spiral and—in advantageous contradistinction with the conventional flat RF-coils—a correspondingly bigger flux of the magnetic field generated by the coil when the driving voltage is applied to its terminals. The loops of the coil 200 in the layers 310A, 310B are otherwise electrically insulated from one another.

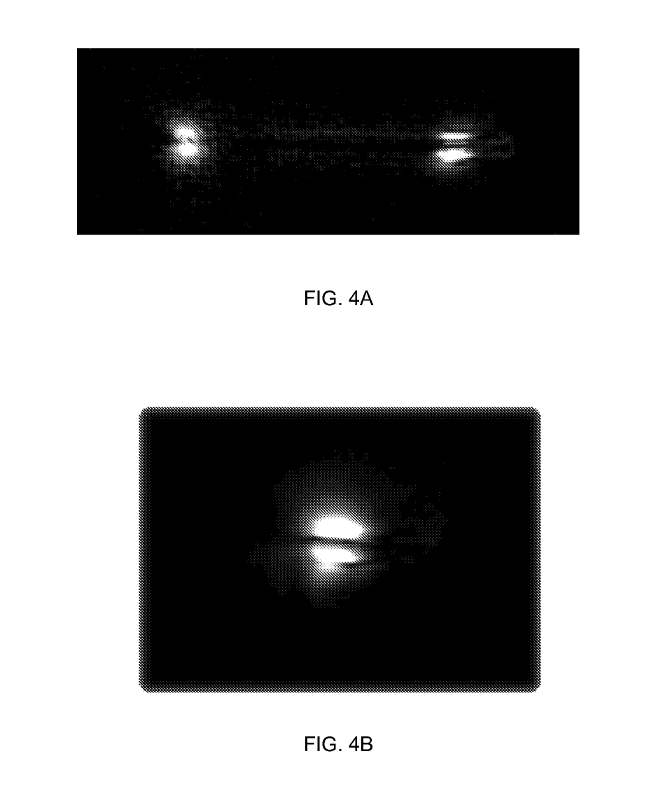

The MRI images of the coils 110, 120 of FIGS. 1A, 1B formed as a result of MRI-tracking experiments when the needle 100 was moved inside a 3 Tesla MRI system, as shown for illustration in FIG. 4A, while a single enlarged MRI image of one of the coils 110, 120 is reiterated in FIG. 4B

According to a related implementation, an MRI-tracking sequence was created and used with the Siemens MRI system. The MRI-tracking system included additional software features which aid in the tracking of these needles. The features improve the ability to detect the position of the RF receiver microcoil, since placement of the coils on a metallic surface results in a received RF signal which is commonly noisier that when such coils are attached to non-conductive and non-metallic surfaces. Specifically, these added features reduce the dephasing of the signal due to the inhomogeneous magnetic field and improve the ability to perform peak detection in the presence of a noisy RF signal. Among these algorithmic features there was program code for performing the MRI-imaging with a very short “time-to-echo” (TE) duration. The TE duration is defined as the time interval between the MRI excitation pulse and the reception of the signal by the receiver of the system. Short TEs are useful in maximizing the detected MR signal because MRI spins close to the metal surface are in an inhomogeneous static magnetic field, B₀, and are therefore readily dephased (i.e. rapidly lose phase coherence between all the spins that are part of the signal).

Additional processing features of the specific implementation included the ability to remove noise from the time-domain traces (“MRI free induction decay” signals), using a combination of signal averaging as well as the use of noise-peak removal algorithms.

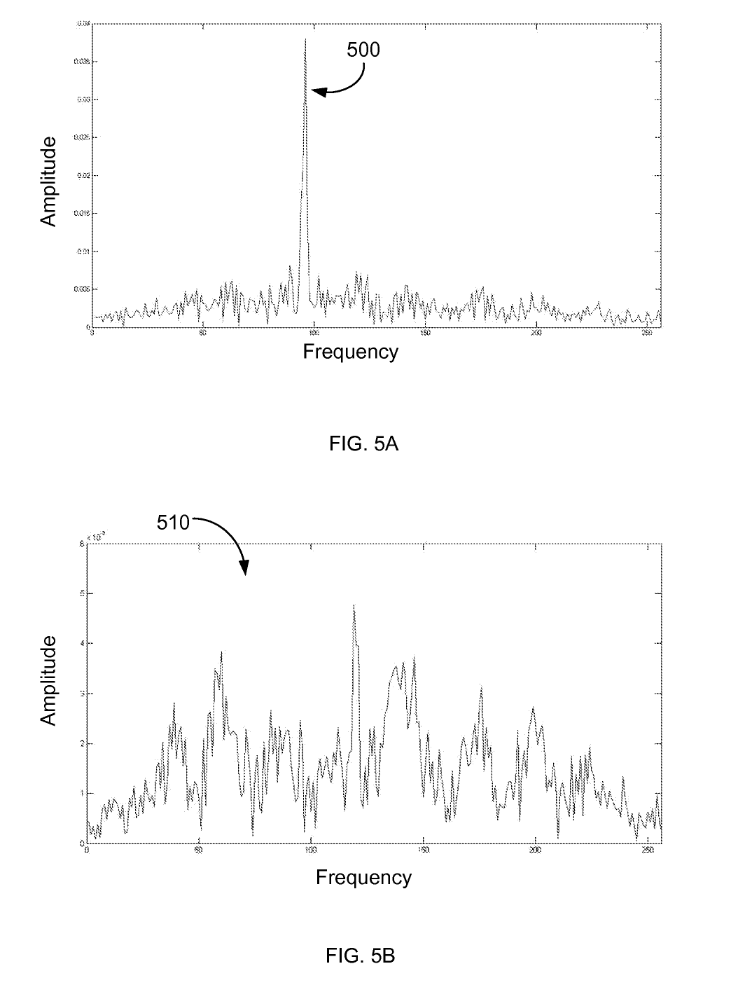

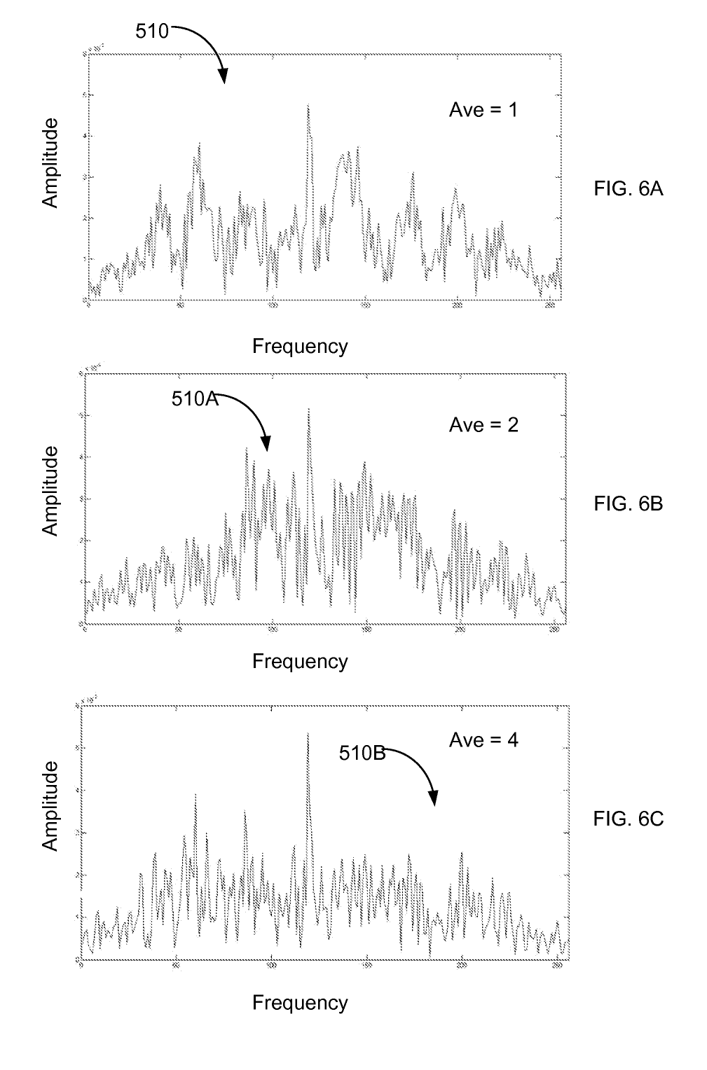

Results of conventionally-implemented MRI-tracking are shown as signals received by the receiver of the MRI-system in FIGS. 5A, 5B, and 6A, 6B, 6C. FIG. 5A is a plot illustrating an MRI tracking signal obtained at a relatively high SNR (˜8), where SNR is herein defined as the ratio between the narrow peak's height and the broader noise baseline clearly presenting a single-peak 500 that is, generally, according to the related art, is not clearly discernible during the conventionally-carried out MRI-tracking of the metallic needles. FIG. 5B illustrates an MRI signal 510 obtained (with the averaging of four sequential acquisitions performed) during the metallic needle tracking procedure at low levels of SNR (˜1.5). The plot 510 contains a multitude of peaks, only one of which is a true peak, while others constitute noise. The plot 510 is a plot typically obtained with embodiments of the related art possessing the shortcomings discussed above. FIG. 6A, 6B, 6C present the MRI tracking signal 510 with signal averaging performed, respectively, over a single trace, over two acquired traces, and over four acquired traces. Signal averaging can be used to reduce the noise and assist in identifying the position of the true peak, but averaging requires time, which reduces the temporal resolution of the tracking process, which may result in an insufficient tracking speed in situations when the anatomy or the device are moving. As would be appreciated by a person of ordinary skill in the art, for these reasons the averaging procedure at low levels of SNR may not be preferred.

In contradistinction to the averaging procedure relied on by the methods of related art, in the embodiment of the invention a time domain noise-peak removal algorithm is used. The noise-based peak manifests as a false-peak on an MRI-trace. The false-peak removal algorithm allows the use of a lower-level signal averaging to detect the true position of the tracking micro-coil. Accordingly, it preserves the degree of temporal resolution of the MRI tracking procedure, which would be reduced if extensive signal-averaging were carried out as required by algorithms of the related art.

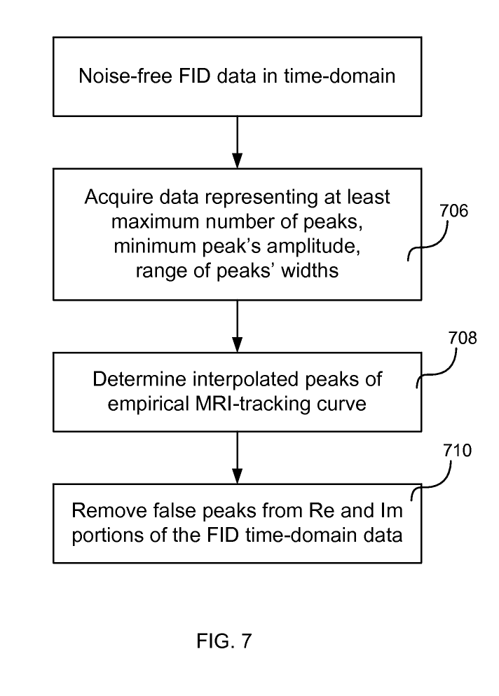

According to the false-peak removal algorithm of the invention, a noise-free Free Induction Decay (FID), a decaying curve representing the MRI tracking signal in a time domain and substantially devoid of sharp peaks, is assumed. The presence of sharp peaks is attributed, therefore, to the presence of noise. In reference to FIG. 7, at step 708, the interpolated peaks are determined with the use of, for example, a quadratic interpolation and based on at least one of a maximum number of peaks, the minimum level of a peak's amplitude, and a range of peak's widths, predetermined at step 706 as input data acquired by a data-processing circuitry of the system of the invention, either via input provided by the user or from a tangible, non-transitory storage medium of the system. At step 710, the false peak(s) are removed, both from the real and imaginary parts of the complex FID time-domain data.

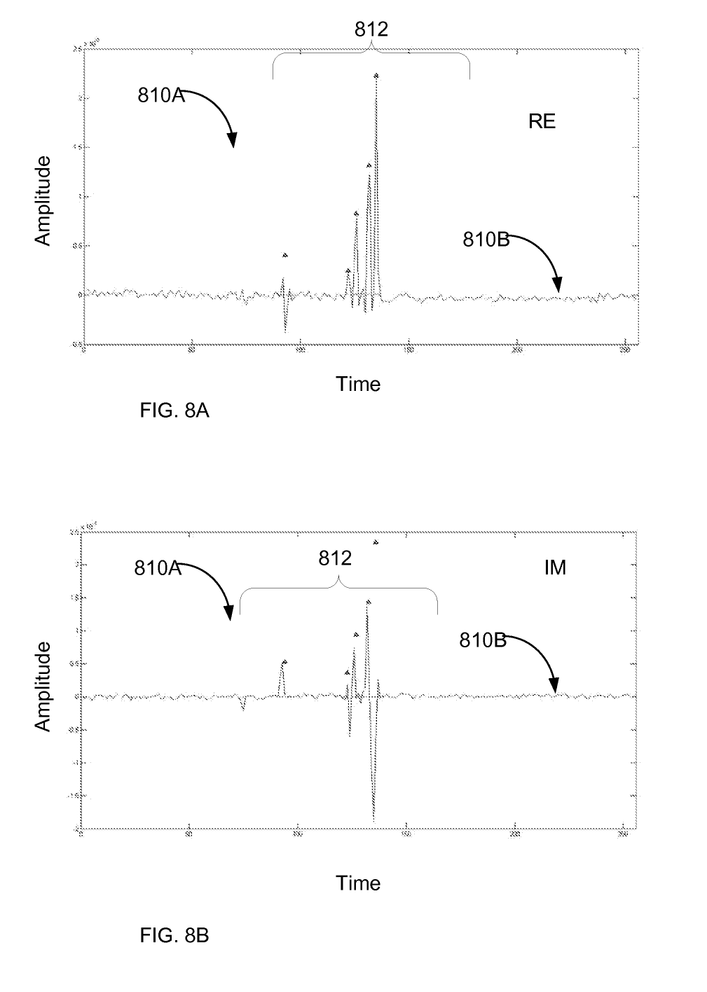

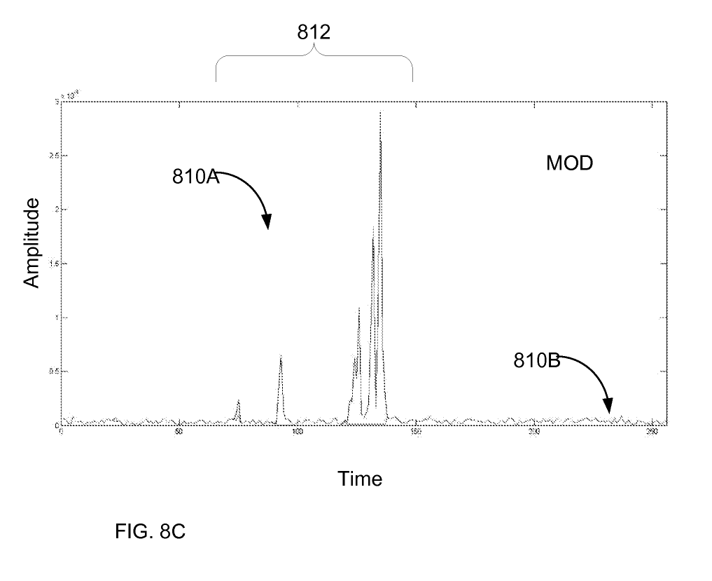

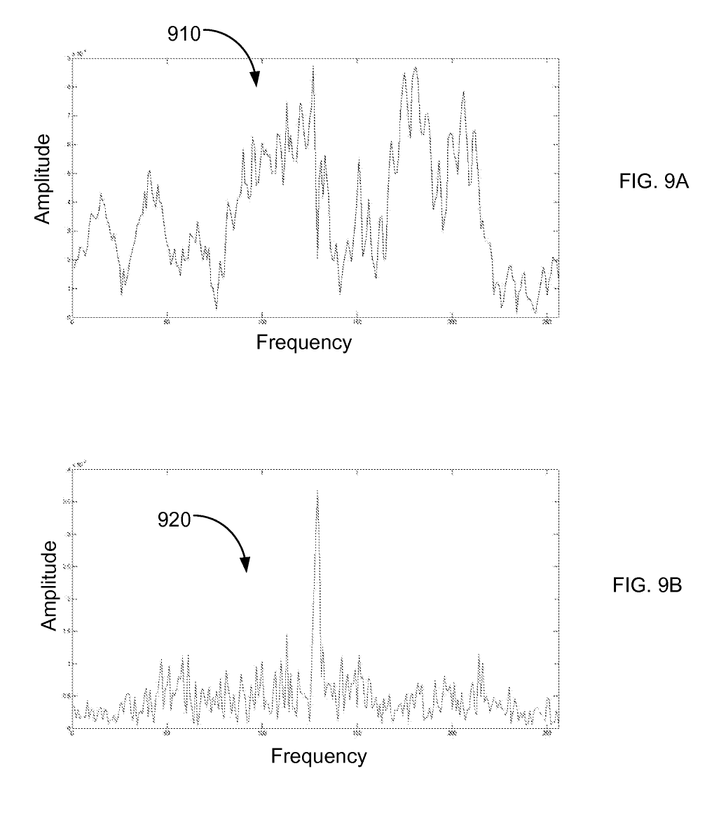

The implementation of the algorithm of FIG. 7 facilitated the ability of the system of the invention to rapidly MRI-track the metallic needles structured according to the embodiment of FIGS. 1A, 1B having coils of the type presented in FIGS. 2, 3A, 3B. FIGS. 8A, 8B illustrate, respectively, real and imaginary parts of a plot 810A representing raw noisy MRI-tracking data that had several detected peaks illustrated by the arrows 812. Trace 810B illustrates the same data after the false peak removal algorithm has been applied. FIG. 8C presents the time-dependence of the absolute value (MOD) of the same data, demonstrating the successful implementation of a false-peak removal algorithm of the invention. Plots 910 and 920 of FIGS. 9A and 9B illustrate in the frequency domain, respectively, a recorded signal at an SNR of 1.2 representing active MRI-tracking of the metallic object equipped with a flat coil according to the embodiment of the invention before and after the false-peak removal filtering algorithm has been applied to the results of tracking.

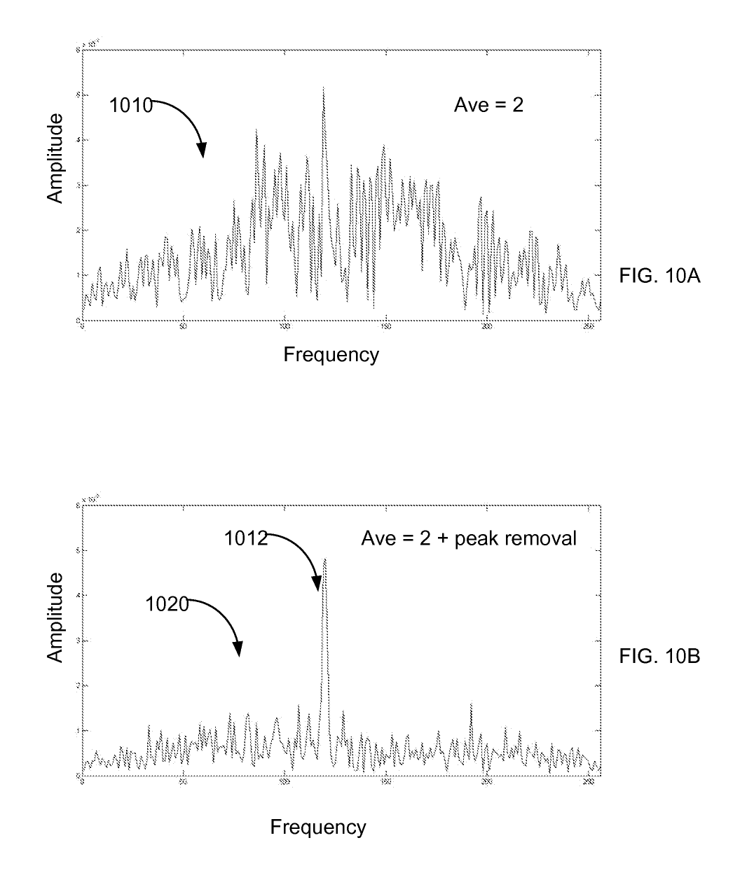

To illustrate clear advantages of the application of a false-peak removal algorithm of the invention over the approaches of the related art, the data shown in FIG. 10A as plot 1010 and collected with active MRI-tracking and averaged over two readings was additionally subjected to a false-peak removal filter of FIG. 7. The clearly-exhibiting a strong true peak 1012 plot 1020 (corresponding to the location of the metallic needle 100 equipped with a coil 110, 120) is presented, for comparison, in FIG. 10B.

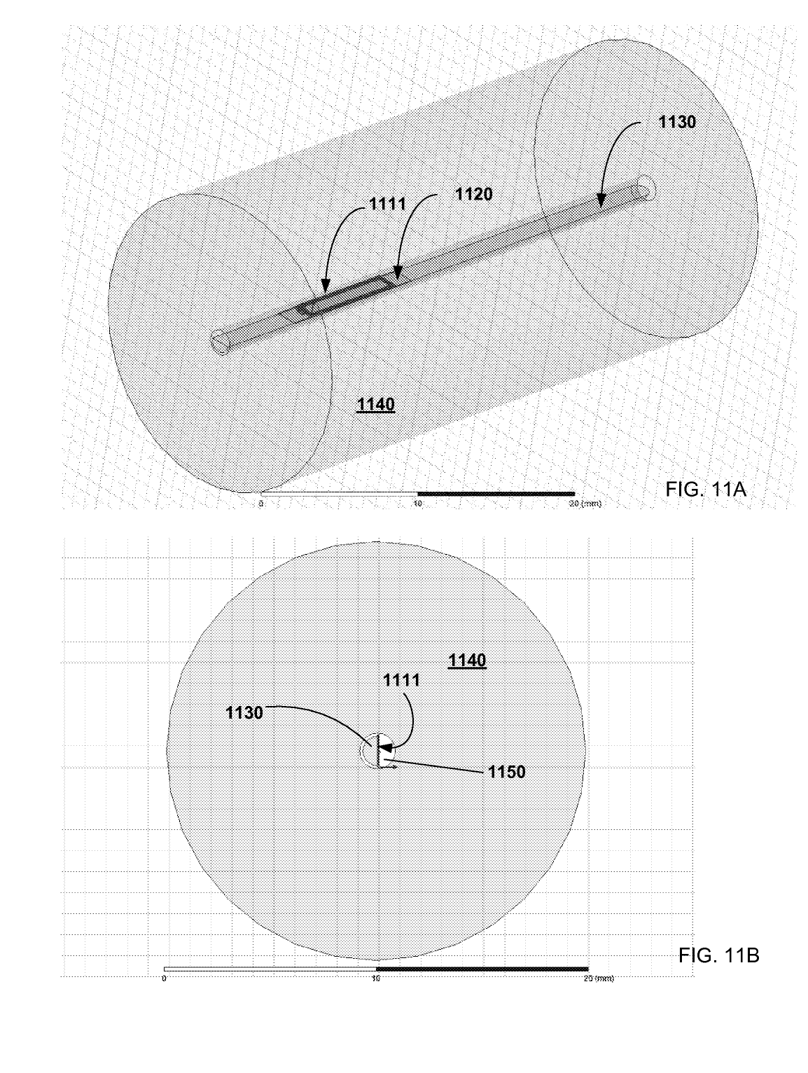

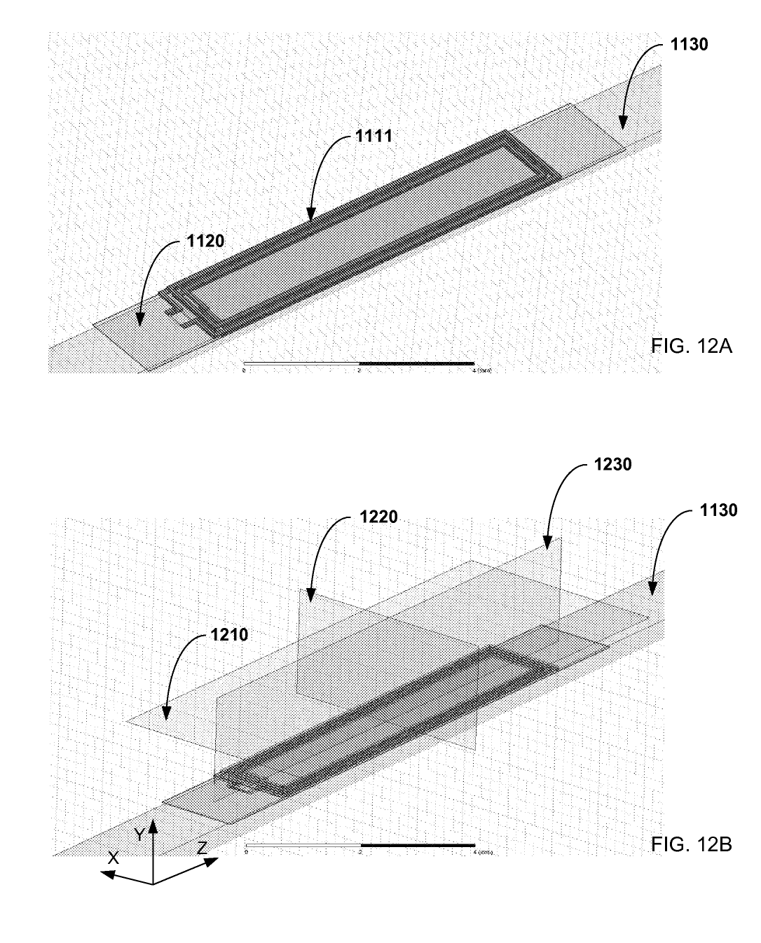

In reference to FIGS. 11A, 11B, 12A, 12B, 13A, 13B, 13C, 14A, and 14B, the electromagnetic (EM) computer simulation of the performance of a single RF coil (such as coils 110, 120 of the embodiment of the invention) was carried out, using a finite-element approach, for a rectangular-loop coil 1111, the two layers of which were structured according to the embodiments 310A, 310B of FIGS. 3A, 3B and which was disposed, on a flexible Teflon substrate 1120, which was then placed on the surface of the semi-cylindrical tungsten needle 1130 as discussed in reference to FIG. 1A, 1B. The ambient medium 1140 (FIGS. 11A, 11B) was simulated as a 0.9% saline solution occupying a 20-mm-diameter cylindrical volume. The 2-mm-diameter cylindrical volume 1150 (defined by the needle's enclosure in practice) in which the needle 1130 was axially disposed along the axis of the volume 1140 was modeled as a volume of vacuum inside the saline solution medium 1140. The coil 1111, viewed as a transmitter/receiver, was considered to be excited with an electrical signal of 1 W at 50 Ohms impedance matching conditions. The electromagnetic (EM) field generated by the so-excited RF coil was calculated at a frequency that is a resonant frequency associated with the MRI-system (in this case, as 123.83 MHZ). The orientation of coronal, axial, and sagittal planes 1210, 1220, 1230 are shown in FIG. 12B. The axial plane 1220 (in FIG. 12B-a plane parallel to the xy-plane) is defined as a plane containing an axis of symmetry of the coil and bisecting the coil into two substantially equal portions perpendicularly to the axis of the needle 1130. The sagittal plane 1230 (in FIG. 12B—a yz-plane) is a plane containing an axis of the needle 1130 and an axis of symmetry of the coil (in FIG. 12B—y-axis) and axially bisecting the coil 1130. The coronal plane 1210 is a plane parallel to the plane of the coil (xz-plane) and positioned about 1 mm above the plane of the coil.

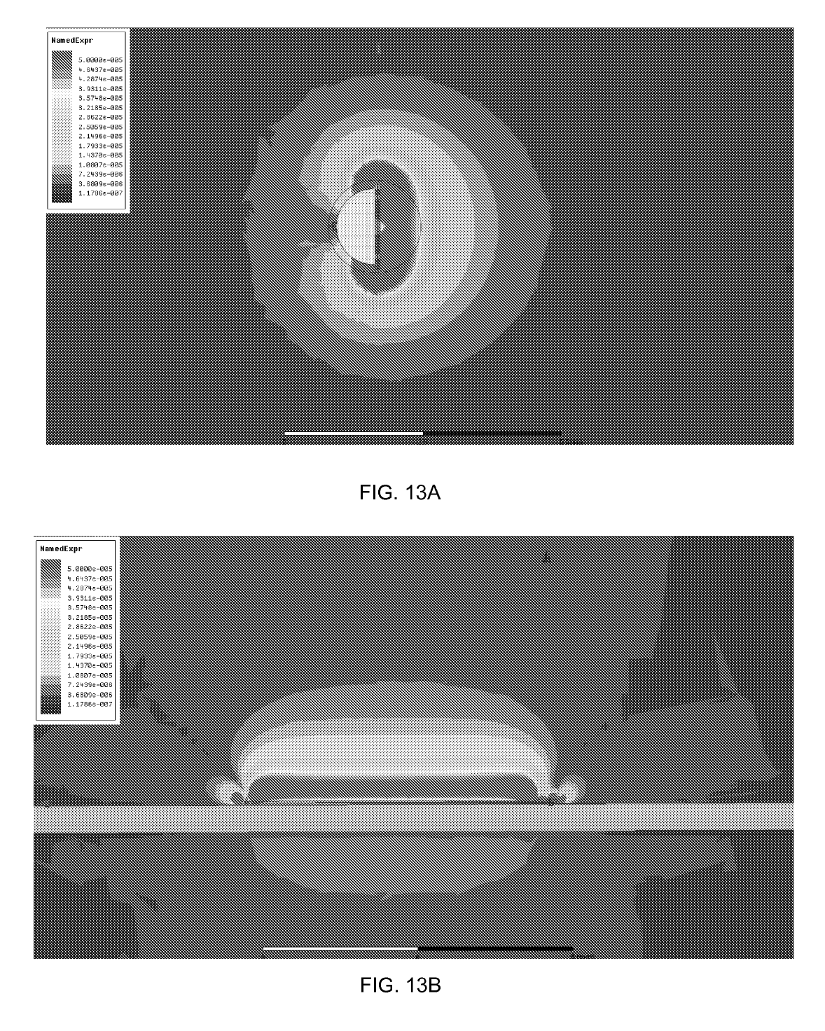

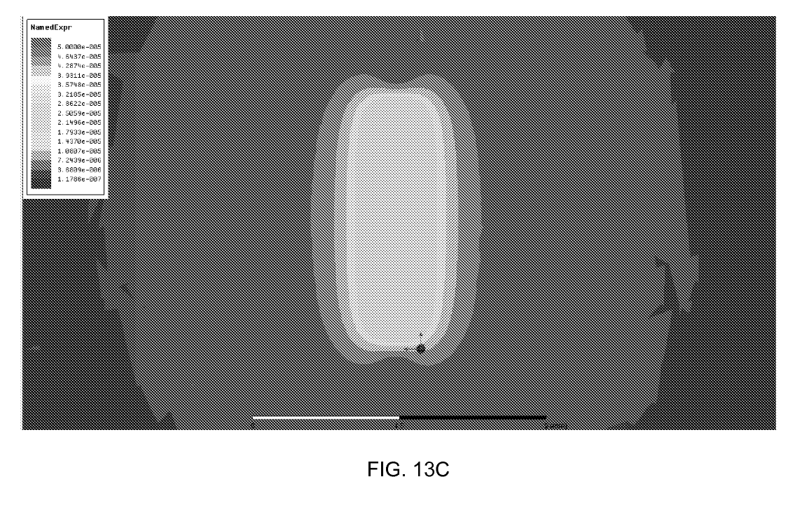

The calculated spatial distribution of the radio-frequency magnetic field (B₁⁻ field), measured in Tesla in the axial plane is shown in FIG. 13A, while FIG. 13B presents the distribution of B₁⁻ in the sagittal plane. The B₁⁻ in the coronal plane is shown in FIG. 13C. The relevance of this information includes the showing of the small penetration of the RF field into the metal itself, which is a cause of eddy currents that, if large, can heat the metal surface upon dissipation.

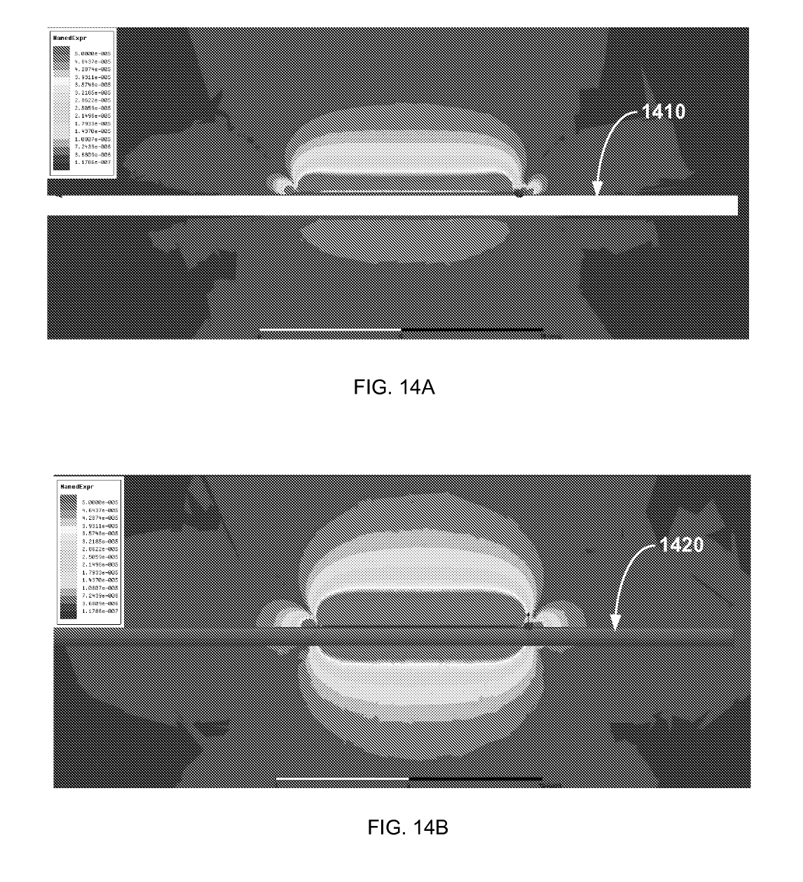

For comparison, FIG. 14A shows a sagittal plane distribution of B₁⁻ for a metallic needle 1410 that has a conductivity that is about 50% lower than that of tungsten, while FIG. 14B shows a sagittal plane distribution of B₁⁻ for a needle 1420 made of FR4, an insulator material frequently used in an electronic circuit boards.

The comparison of the magnetic field distributions of FIGS. 13B, 14A, 14B in the sagittal plane illustrates the operational advantage of use of the embodiment of the present invention. In particular, the RF coil employed with an embodiment of a metallic needle (FIGS. 13B, 14A) projects the field in a direction that is primarily orthogonal to the surface of the tracked metallic object. Placement of this coil on a non-metallic surface (FIG. 14B) would result in a RF pattern that projected further from the surface, but the distance outward projected by the coil on a metallic surface (FIG. 13B) is sufficient for the tracking purposes, and in addition, this pattern is not very sensitive to the conductivity values of the metallic surface (FIG. 14A), as seen from the similarity between FIGS. 13B and 14A.

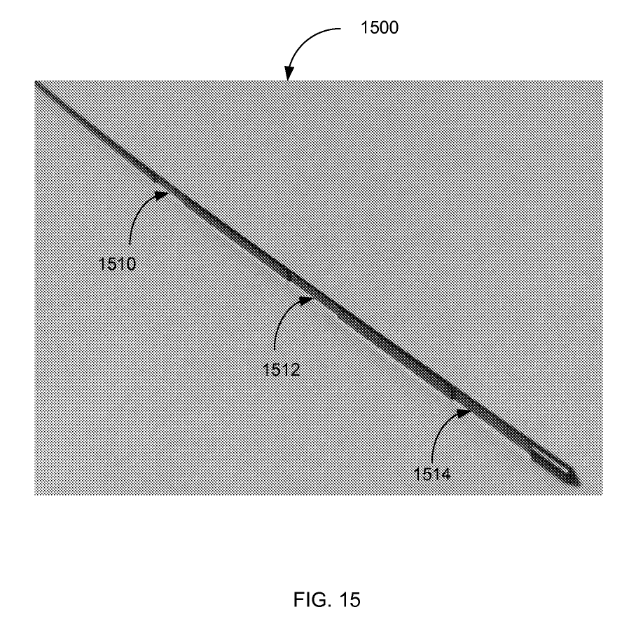

In implementing embodiments of the present invention, the ability of the MRI-system to actively track metallic brachytherapy needles equipped with a flat RF-receiver coil-on-a-flexible-substrate was demonstrated. Modification of a commercial brachytherapy needle to implement an embodiment of the invention includes reducing its diameter at the specific points on the shaft which are to be tracked, to facilitate the juxtaposition between the RF-coil-carrying circuit board to the needle, thus enabling active MRI-based tracking of the needle without increasing the diameter of the needle. The diagram illustrating such needle 1500 with three slots 1510, 1512, 1514 (that are used for attaching three printed circuit coils) is presented in FIG. 15.

### Examples of Composite Implementations Employing Multiple Materials.

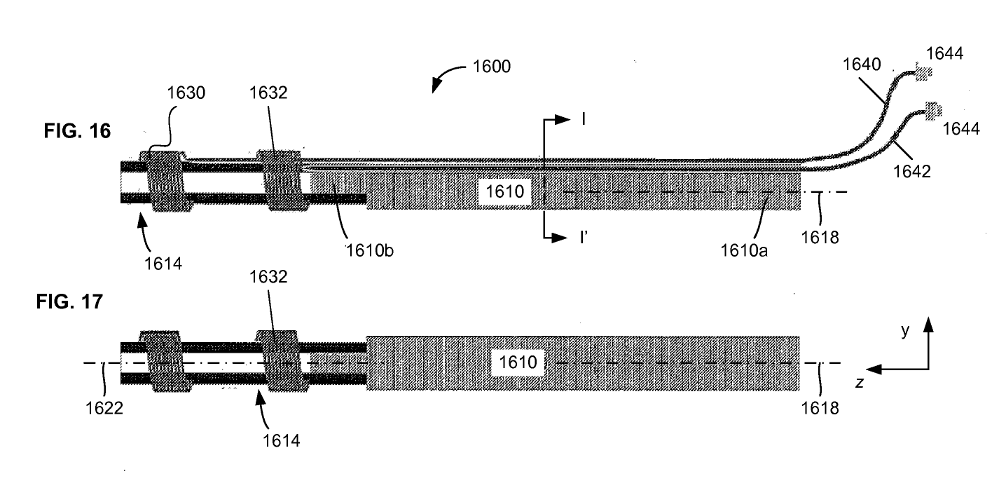

FIGS. 16 and 17 illustrate schematically, in side plan views, an embodiment 1600 of a needle system according to the idea of the invention. The embodiment includes a first body portion 1610 made of metal and having proximal and distal ends 1610a, 1610b and a second body portion 1614 including a strong non-metallic and diamagnetic composite material such as carbon fiber, and is bonded onto the distal end 1610b. The ratio of lengths of the first and second body portions is preferably at least 10 or higher. For example, while a first length value of the first body portion is about 12 inches, a second length value characterizing the second body portion is about 1.2 inches. As the overwhelming majority of the overall length of the needle 1600 retains its metallic properties, in practice the entire length of the needle remains trackable with the use of active and passive methods compatible with the MRI procedure, thereby providing a clinician with accurate detection data representing the position and orientation of the needle to arrive at an informed decision about the need to further reposition the needle.

The second body portion 1614 is tubular, with the ratio of inner diameter, ID₁₁₄, to the outer diameter, OD₁₁₄ of about ID₁₁₄/OD₁₁₄=½. In one example, ID₁₁₄ is about 0.020″ while the OD₁₁₄ is about 0.039″. The first body portion 1610 is shaped as a solid metallic (e.g., tungsten alloy) cylinder with the OD₁₁₀ of about 0.054″, with the exception of the distal end 1610b that is appropriately machined to remove a part of the solid cylinder and create a cross-sectional profile substantially matching the ID₁₁₄. Accordingly, a portion of about 1.25″ of the distal end 1610a of the needle has a cross-section, formed in a plane substantially perpendicular to an axis 1618 of the portion 110, which has a dimension of OD₁₁₄ less applicable machining tolerances to ensure friction fit between the first and second body portions.

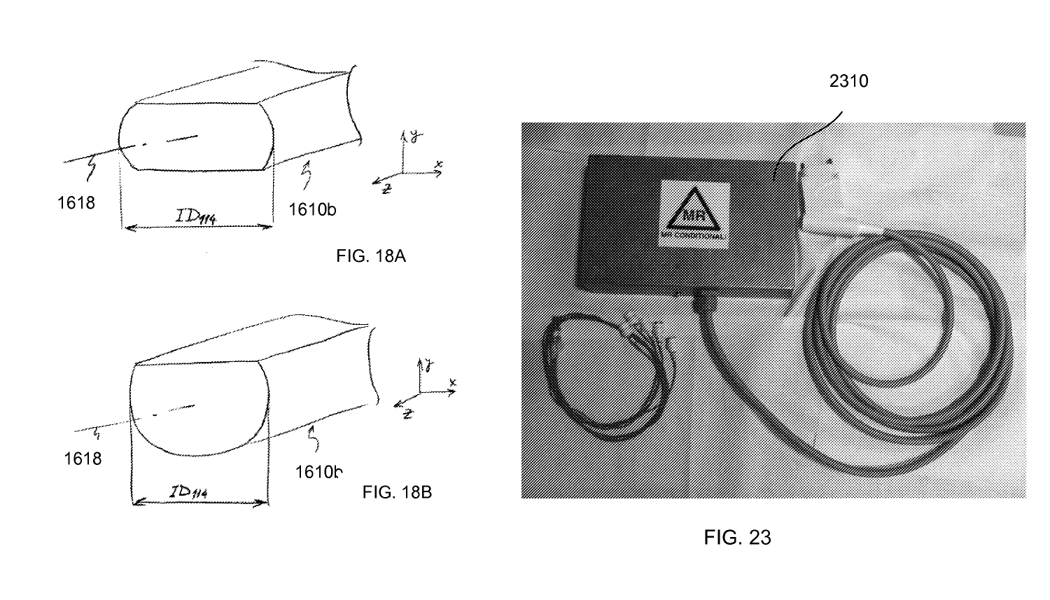

In one implementation, for example, the distal end 1610b is dimensioned as a solid cylinder that is substantially co-axial with the proximal end 1610a. In another implementation, the distal end 1610b is shaped as a cut cylinder, truncated with a plane parallel to the axis 1618 at least on one side of the axis 1618. The examples of so-formatted distal end 1610b are shown schematically in FIGS. 18A, 18B. The coupling between the portions 1614 and 1610 is enabled by tight fitting of the female end of the second portion 1614 over the male distal end 1610b, optionally complemented with epoxy adhesive. In a related implementation, the coupling region may be reinforced by wrapping the coupling region with epoxy-infused thread or wire (for example, a Kevlar cord or multiple loops of the wire comprising the microcoils 1630, 1632). It is appreciated that, while the shaping of the distal end 1610b is shown in FIGS. 16, 17 to substantially preserve the axial symmetry of the first portion 1610 (such that the distal end 1610b remains extended substantially along the axis 1618), in a related embodiment (not shown) the distal end 1610b can be structured as inclined with respect to the axis 1618. In such a case, when brought in operational contact with each other, the axes 1618 and 1622 of the first and second portions 1610, 1614, as observed in a view of FIG. 16, would not remain parallel and form an angle with respect to one another.

The second tubular portion 1614 carries a plurality (as shown—two) microcoils 1630, 1632 wrapped around it and connected by respective electrically conductive leads 1640, 1642 with the connector-plugs 1644 and through the connector-lugs—with external circuitry (not shown). As compared with FIG. 16, FIG. 17 illustrates the embodiment 1600 without the lead 1640, 1642. In one example, each of the microcoils 1630, 1632 is defined by 10 turns of a 38 AWG magnet wire, and the electrically-conductive leads include co-axial cables (46 AWG, 50 Ohm) allowing, in operation, the detection of the position of the needle tip 1614.

Modifications to, and variations of, the illustrated embodiments may be made without departing from the inventive concepts disclosed herein. For example, in a related embodiment the first portion 1610 may contain at least one lumen segment extending there through and fluidly connecting the hollow of the second portion 1614 with the proximal end 1610b.

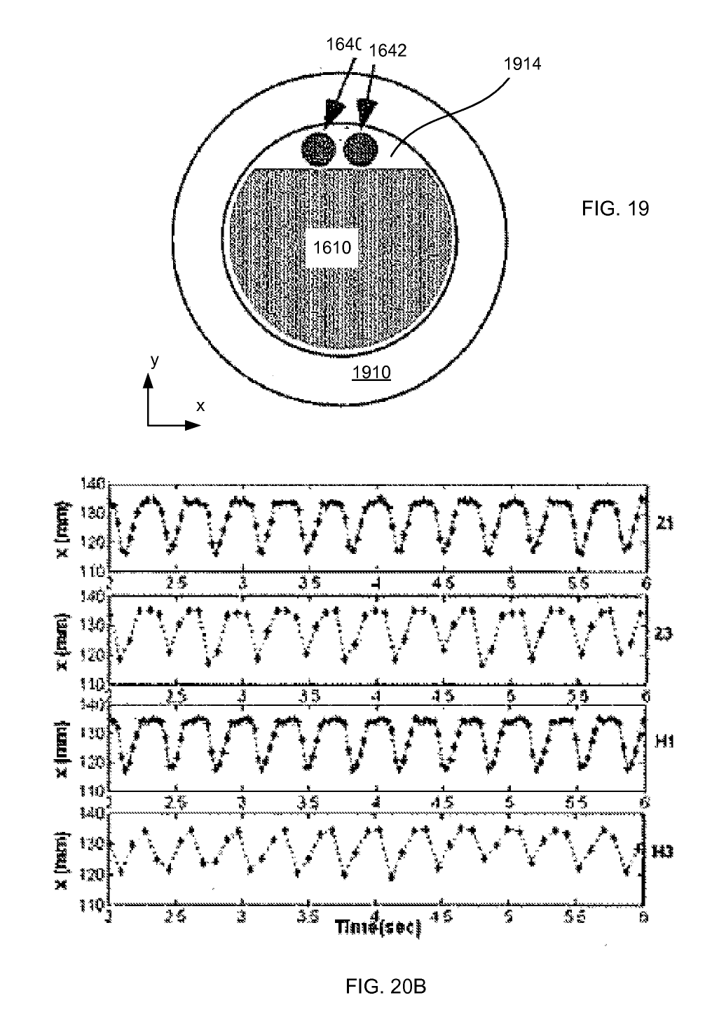

Referring now to FIG. 19, the embodiment 1600 of FIG. 16 is shown disposed inside a catheter 1910 in a plane I-I′ that is substantially perpendicular to the axis 1618. An example of the appropriate catheter is provided by a plastic sharp-tipped catheter of Oncosmart ProGuide Needles (Nucletron, Inc, Best, Holland), with the OD₄₁₀=0.075″, ID₄₁₀=0.056″, and length of about 9.5″. To accommodate the positioning of the conductive leads 1640, 1642 along the first portion 1610 under the catheter, a section of the first portion 1610 is carved out to form a gap 1914.

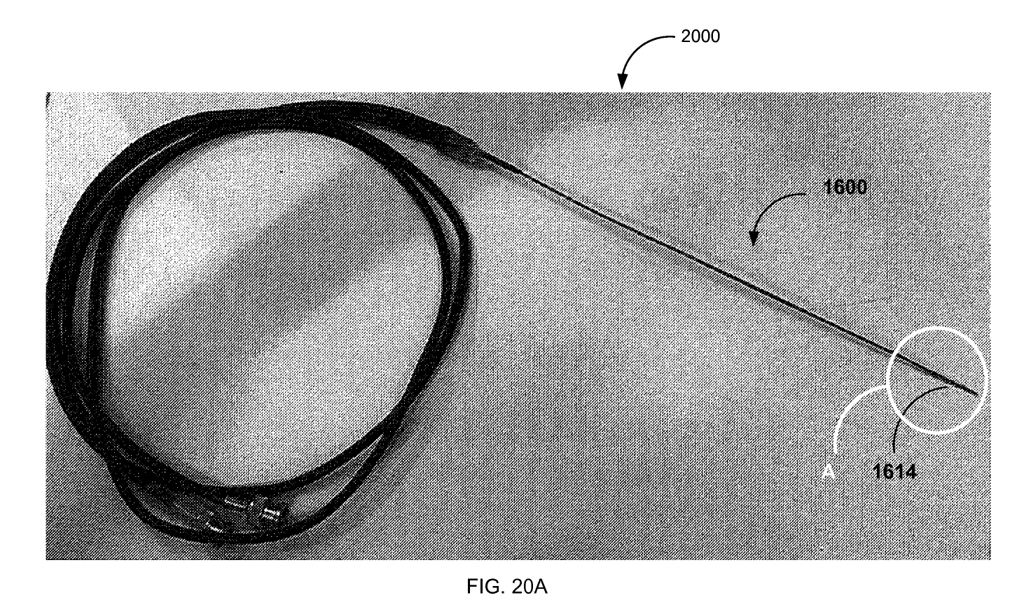

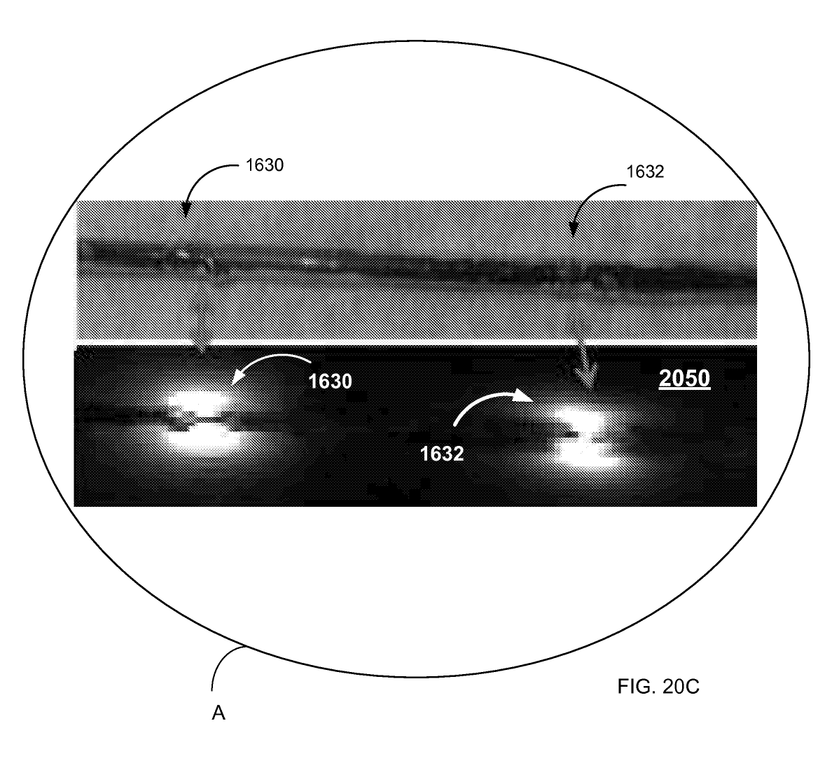

FIG. 20A provides a perspective view of a practical implementation 2000 of the device of the invention, formatted as a trackable high-dose brachytherapy needle for use in a 3T Siemens Verio system. The test of the embodiment was carried out during rapid (about 8 Hz) motions of the assembly over distances of about 20 mm to about 40 mm. Empirical data, obtained with testing the device 2000 accompanied with phantoms including 5 adjacent needles placed in the tissue in close proximity to the actively tracked needle 1600, evidenced successful active tracking of the needle 1600 with spatial resolution of about 0.6-by-0.6-by-0.6 mm³ at a rapid rate of about 20 to about 40 frames per second despite the in-homogeneity in RF magnetic field created by the neighboring needles. FIG. 20B is a graph representing MR-tracking recording of 18 mm amplitude, 3 Hz oscillatory motion of the needle assembly. (Such motion of the needle, in practice, may be caused, for example, by the physiological motions of the tissue in which the needle is embedded. As such, the image of the needle acquired regularly is blurred, as the imaging data are taken in a frame of reference associated with the MRI equipment. The active tracking of the needle device according to the present method results in determination of the absolute position of the needle device and/or of its component, and facilitates the mapping or translation of the imaging data from the frame of reference associated with the MRI equipment to the frame of reference associated with the needle device. In such needle-related frame of reference, the final composite image of the needle is substantially free of artifacts caused by the physiological motion of the tissue.) Here, “Z” curves represent the tracking results in the absence of motion for different spatial positions with the use zero-phase difference, and “H” curves represent the tracking results with the use of a Hadamard multiplexing MRI-tracking scheme with different phase-field dithering (PDF) cycle lengths denoted as “1” and “3”. Embodiments of the invention were also tested under X-fluoroscopy to provide evidence of accurate imaging of the locations of the carbon fiber based second portion 1614 and the metal based first portion 1610 of the embodiment under X-rays. FIG. 20C combines the depiction of the distal end 1614 (enlarged) of the embodiment of FIG. 20A and an MR image 2050 identifying the two microcoils 1630, 1632.

The methods employed in tracking the needle 1600 included specific active tracking sequences that facilitate minimization of errors in determining the absolute position of the device by comparing the actively-detected position with that determined from high-resolution MRI images, that facilitates, in practice, more frequent update of empirical data representing the position of the needle, and that provide real-time input to a clinician enabling him to precisely advance the needle in the biological tissue.

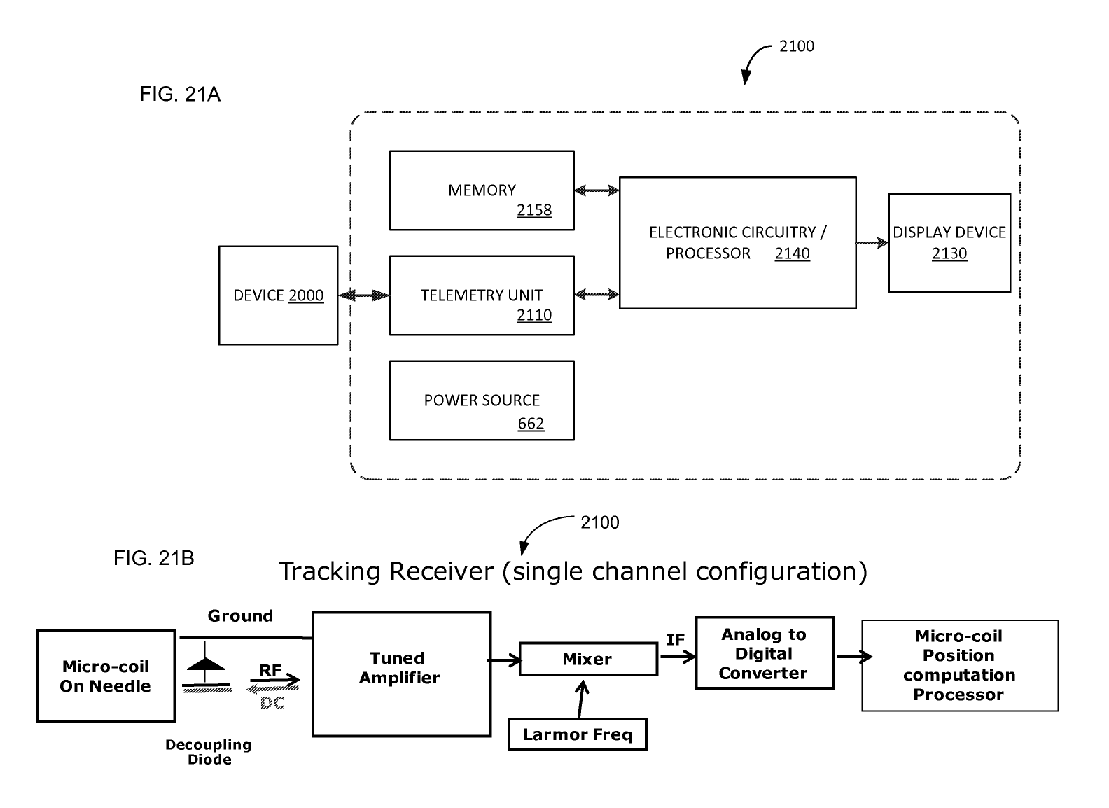

In reference to FIGS. 21A and 21B, illustrating schematically the system 2100 of the invention, the device 2000 is complemented with a specific tracking-coil interface or telemetry unit 2110 enabling the active tracking to be concurrently implemented on multiple microcoils (for example, up to eight) with minimal latency (less than about 100 frames per second) between tracking acquisition of data and the output of the tracking data representing location and/or orientation of the needle through the output of the tracking system of the invention toward external devices.

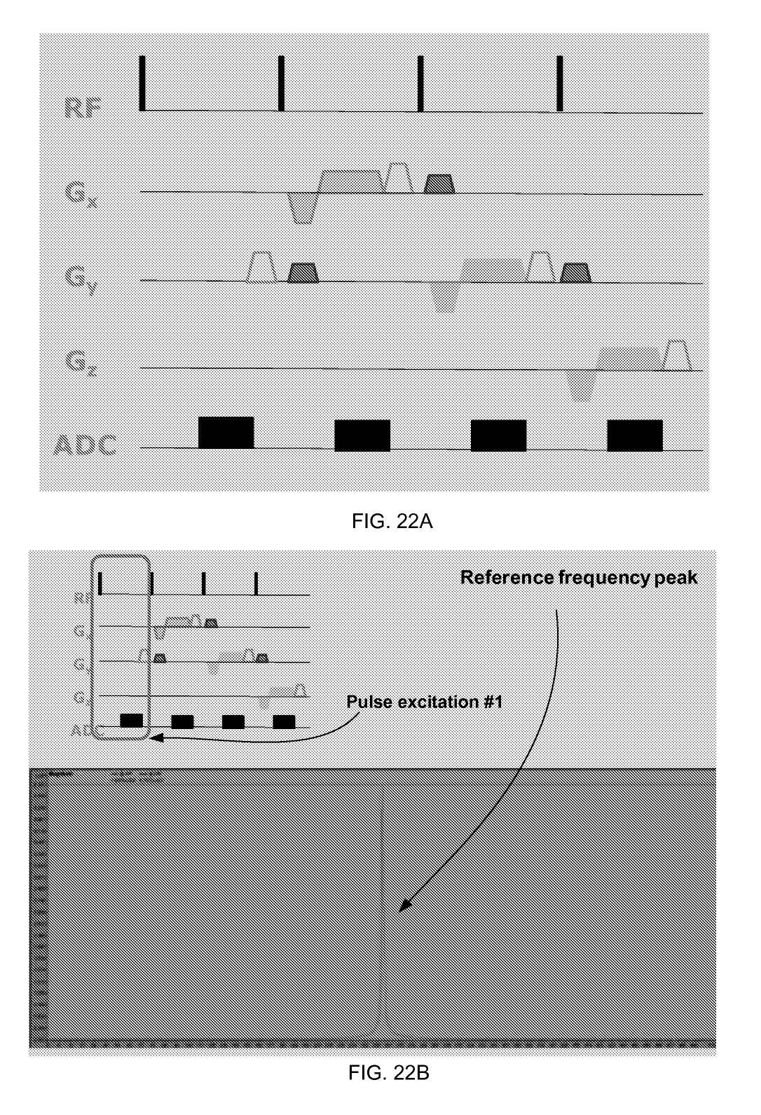

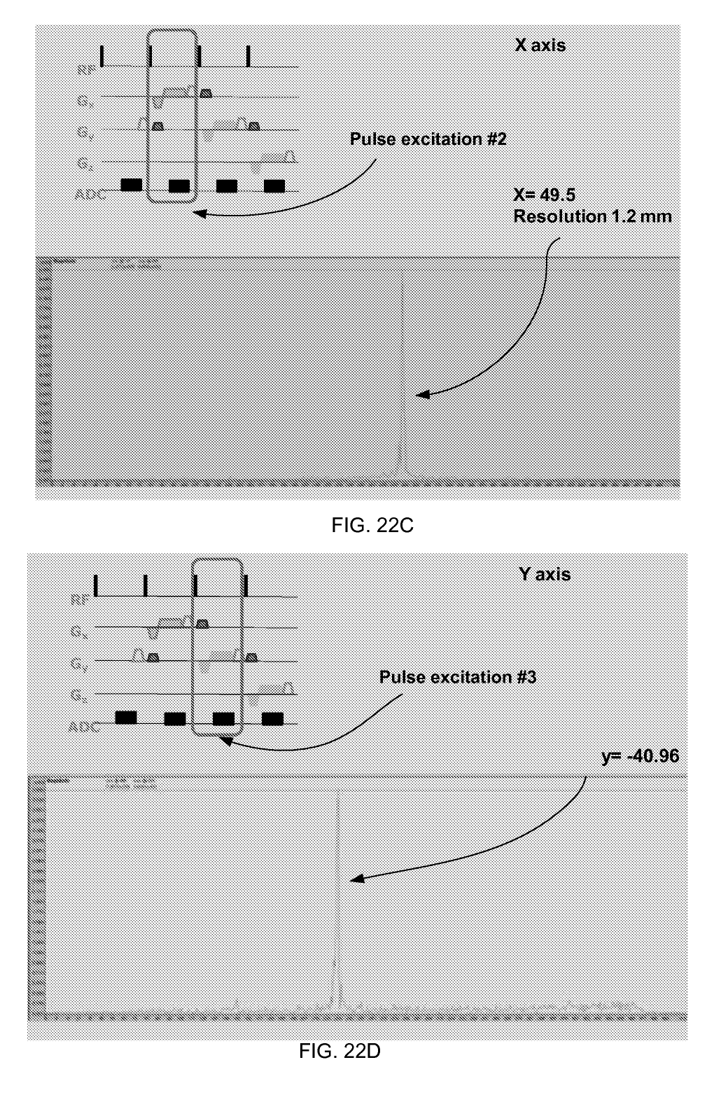

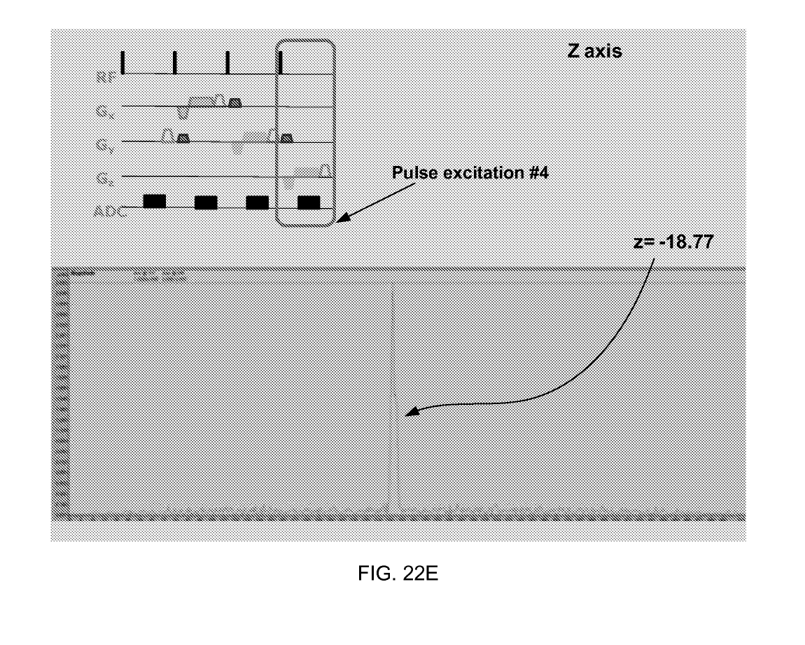

This tracking interface 2110 (illustrated in more detail in FIG. 21B) is similar to multichannel radio-frequency receiver coil interfaces provided by the MRI vendors, with added provisions for patient safety. The additions to the tracking interface of the invention are necessitated by the fact that such interface is generally placed within the field generated by the MRI system, and because it collects signals from invasive interventional devices. The major components of this tracking interface include, for each acquisition channel (i.e., each microcoil): (1) a radio-frequency (RF) amplifier tuned to the MRI scanner's Larmor frequency; and (2) a Direct Current (DC) voltage output, which is used to actively decouple pin-diodes during RF pulse transmission and thus prevent high-voltages from being received by the receiver. Since the needles are invasive devices, the interface must satisfy the leakage-current and high-voltage regulatory standards for invasive devices. One way to satisfy these standards is to power this interface with an isolated power supply, or use a battery source. After signal amplification, the MRI signals originating in the needle are phase-sensitively demodulated by being mixed with a Larmor frequency wave which originates from the scanner's main RF-transmission amplifier. Once demodulation is performed, the resulting lower-frequency signals are digitized. The demodulated digital signals are then transmitted to a reconstruction computer. Such transmission can be performed over coaxial cables, fiber-optic cables, or Wi-Fi channels. In the reconstruction computer, the signals are processed to provide the three dimensional positions of each microcoil. These positions are then processed to provide the microcoil and needle positions and orientations, which are displayed to the clinicians while they are navigating the needles through the patient anatomy. The positions can also be fed back to the scanner in order that it acquire MRI data close to the needle tip, thereby providing data on tissues surrounding the tip, which may enable the clinician to choose the optimal path to advance the needle in order to reach the target position with minimal risk to eloquent structures along the way. To obtain the position of each of the microcoils, dedicated MRI-pulse sequences (“MR-tracking sequences”) are utilized. The MR-tracking sequences acquire three one-dimensional projections along three orthogonal spatial axes (x,y,z). In reference to FIGS. 22A, 22B, 22C, 22D and 22E and Tables 1 and 2, zero-phase-reference and Hadamard multiplexing schemes can be used to suppress the effects of static magnetic field (B₀) in-homogeneities. Parameters of the modulation schemes are summarized in Tables 1 and 2. The phase-field dithering method is also employed by applying multiple orthogonal magnetic-field gradients in arbitrary directions and selecting the highest-quality tracking signal based on certain criteria. FIG. 21A provides an overview of one implementation of the MR-tracking pulse sequence, referred to as the “zero-phase reference” approach, which is required for the acquisition of the three-dimensional (X,Y, Z) position of the micro-coils. The “zero-phase-reference” approach includes four pulse sub-sequences which are played out sequentially. (The “Hadamard approach” that is discussed below, also utilizes four pulse sub-sequences, but it follows a different encoding scheme). FIGS. 22B through 22E provide the detailed MR tracking pulse sub-sequence diagrams, which are used to acquire a correction for the magnetic field in-homogeneity (FIG. 22B), then the X position of a micro-coil (FIG. 22C), followed by the determination of the Y position of the micro-coil (FIG. 22D). FIG. 2E illustrates the determination of the Z position of the micro-coil. It is appreciated that precise determination of the 3D positioning of a micro-coil of the needle that is not affected by the physiological motion of the tissue is enabled by the presently employed embodiment of an active-tracking method.

FIGS. 22A through 22E and Tables 1, 2 also illustrate an example of addition to the fundamental MR-tracking sequences of dephasing gradients, which are added to six directions on a plane orthogonal to the readout direction. The addition of phase-dithering to the basic MR-tracking pulse sequences can improve the quality of tracking of the needle device when the tracking SNR is low, and compensates the radio-frequency in-homogeneity (B₁) effects as well, although it does require performing a greater number of sub-sequences (more than the minimal number of four sub-sequences) in order to acquire the 3D position of a micro-coil. Consequently, the use of phase-dithering in the embodiment of the invention facilitates the reduction of the temporal rate (or speed) of active tracking of a device within the field-of-view of the MRI scanner.

FIG. 23 is an illustration of practical implementation of the system of the invention, showing the eight-receiver tracking module 2310.

TABLE 1
Zero-phase-reference scheme for acquiring a three dimensional
position of a microcoil. The forms of pulse excitations corresponding
to each of the sub-sequences are shown in FIG. 22A.

| Excitation | X | Y | Z |
|---|---|---|---|
| 1 | 0 | 0 | 0 |
| 2 | 1 | 0 | 0 |
| 3 | 0 | 1 | 0 |
| 4 | 0 | 0 | 1 |

TABLE 2
Hadamard multiplexing scheme for acquiring the three-dimensional
position of a microcoil. The four excitations used in Hadamard
replace the four sub-sequences shown in FIG. 22A with excitations
each of which is a linear combination of the basic “zero-phase-
reference” sub-sequences. Accordingly, the X, Y, and Z positions
of a microcoil can be determined by this method as well.

| Excitation | X | Y | Z |
|---|---|---|---|
| 1 | −1 | −1 | −1 |
| 2 | 1 | 1 | −1 |
| 3 | 1 | −1 | 1 |
| 4 | −1 | 1 | 1 |

With the zero-phase-reference scheme, a reference frequency offset is provided by the excitation without a spatial encoding gradient. The X, Y, and Z positions are determined by subtracting the location of the reference-frequency peak from the peak location provided by the Fourier-transformed signal of the each of the directionally encoded profiles. With the Hadamard scheme, the positions are calculated by taking the linear combinations of the peak positions computed from each excitation.

Irrespective of the excitation method used (whether the zero phase reference or Hadamard), the signal profile obtained in association with each pulse excitation depends on the orientation of the coil with respect to the frequency encoding direction utilized in this specific excitation. As a result, the following centroid algorithm is used to find the position of the microcoil: (1) the location Lₘₐₓ of the maximum signal intensity is found; (2) a window W of twice the length of the microcoil, with the window center at Lₘₐₓ is set; (3) The location of the coil L_{C} is calculated to be the centroid of the signal intensity profile within the window:

$${L}_{c} = {\sum }_{{L}_{m} - W / 2}^{{L}_{m} + W / 2} l \cdot S ( l ) / {\sum }_{{L}_{m} - W / 2}^{{L}_{m} + W / 2} S ( l )$$

where S(l) is the signal intensity at location l.

The orientation of the needle device 2000 of FIG. 20 can be calculated by using the positional information provided by multiple (at least two) coils. The tip position of the needle can then be computed by extrapolating along the vector connecting two (or more) microcoils. Both the position and orientation information are transferred to a graphical workstation such as the device 630 for visualization. Low-pass filtering in the time domain is performed on the positional data to reduce positional spatial “jitter” before it is sent to the display. Reducing this “jitter” aids in practical use of the needle tip position by the clinician.

The MR tracking sequence and reconstruction method are currently implemented on the Siemens MRI acquisition and reconstruction engine, but they can be implemented on stand-alone modules as well.

In further reference to FIG. 21A, the system 2100 further includes a pre-programmed electronic circuitry (in a specific implementation it may be a computer processor) 2140 governing the operation of the needle tracking, collecting the data representing the needle position and cooperating such data with data representing MR images provided by the MRI system to create a visually perceivable representation of the needle 1600, on a display device 2130 (optionally—overlapped with at least one MR image). The processor 2140 may be realized by one or more microprocessors, digital signal processors (DSPs), Application-Specific Integrated Circuits (ASIC), Field-Programmable Gate Arrays (FPGA), or other equivalent integrated or discrete logic circuitry. Programming information may be received from an external clinician programmer or an external patient programmer. When implemented wirelessly, the telemetry unit 2110 may receive and send information via radio frequency (RF) communication or proximal inductive interaction of a programmer.

A tangible non-transitory computer-readable memory 2158 may be provided to store instructions for execution by the programmable electronic circuitry 2140 to control the pulse generator 2144 and the switch matrix 2156. For example, the memory 2158 may be used to store programs defining different sets of pulse parameters and microcoil combinations. Other information relating to operation of the system 2100 may also be stored. The memory 2158 may include any form of computer-readable media such as random access memory (RAM), read only memory (ROM), electronically programmable memory (EPROM or EEPROM), flash memory, or any combination thereof.

A power source 2162 delivers operating power to the components of the system 2100. The power source 2162 may include a rechargeable or non-rechargeable battery or an isolated power generation circuit to produce the operating power.

It is appreciated that embodiment(s) of the invention enable rapid advancing of needles in interventional procedures and real-time visualization of the needle tip with respect to the internal patient anatomy. The tracking data representing location and trajectory of the needle can be overlaid on pre-acquired MR image(s) and used to control the MRI imaging location and orientation thereby further improving real-time navigational guidance. The latter is of particular importance in a situation where advancing the needle may result in a dynamic displacement of the tissue or change/rupture of the tissue boundaries. When used in conjunction with the embodiment of the invention, multi-channel MR-tracking receivers (which can be configured as part of an MRI scanner) allow simultaneous tracking of a multiplicity of needles configured according to the invention—for example, an array of such needles optionally closely spaced from one another. When used in conjunction with a plastic enclosure (such as the plastic tubing 1910 of FIG. 19), embodiments ensure adequate sterility of the procedure, thereby reducing the risk of infection of the tissue structures. Envisioned commercial applications of the embodiments include MRI-guided cancer radiation brachytherapy applications (for example, cervical, prostate, head and neck tumors); MRI-guided biopsy applications (breast, prostate, head and neck tumors; abdominal tumors); MRI-guided therapy (thermal or cryo ablative); as well as MRI-guided vascular interventions (e.g., neurovascular, cardiovascular, peripheral).

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of these phrases and similar language throughout this specification may, but do not necessarily, all refer to the same embodiment. It is to be understood that no portion of disclosure, taken on its own and in possible connection with a figure, is intended to provide a complete description of all features of the invention.

Also, features of the invention are described with reference to corresponding drawings, in which like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

The invention as recited in claims appended to this disclosure is intended to be assessed in light of the disclosure as a whole.

Modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

1. A device adapted for use with a system for actively tracking of a position of a device within a magnetic resonance imaging (MRI) scanner, said device comprising:
a fully-metallic filament extended along an axis and including proximal and distal ends, a first length, and extended along an axis, said filament having a substantially flat surface along the first length; and
at least one MR receiver coil including at least one first loop that forms a first electrically-conductive trace disposed in a first plane parallel to the substantially flat surface such that a normal to said plane is transverse to the substantially flat surface, said at least one coil having electrical terminals electrically extended towards the proximal end.

2. A device according to claim 1, wherein said at least one coil further includes at least one second loop that forms a second electrically-conductive trace disposed in a second plane parallel to the substantially flat surface and electrically connected to the at least one first loop such as to define a length of the at least one coil as a sum of lengths of the first and second electrically-conductive traces, the first and second planes being different and parallel to one another.

3. A device according to claim 1, in which the needle further comprises a plastic sheath encasing at least the fully-metallic filament, wherein the first plastic portion and the plastic sheath are dimensioned to form a gap there between, and wherein an electrical extension of a terminal toward the proximal end is disposed in the gap

4. A method for fabricating an interventional needle device, the method comprising:
attaching a tubular distal needle segment, made of a material that is non-metallic and either paramagnetic or diamagnetic, to a distal end of the carrying portion of the needle made of metal;
disposing at least one coil of electrical conductor adjacent to the tubular distal needle segment; and
providing an electrical output to at least one coil.

5. A method according to claim 4, wherein a ratio of a first value representing a length of the carrying portion and a second value representing a length of the tubular distal needle segment is at least 10.

6. A method according to claim 4, wherein the attaching includes dimensioning the distal end of the carrying portion to ensure friction fit of the distal end inside the tubular needle element.

7. A method according to claim 4, further comprising
providing an electrically conducting member between the coil and the electrical output; and
encasing at least the carrying portion of the needle in a plastic tubing to pass the electrically conducting member inside the tubing.

8. A method for using a tracking system for magnetic resonance imaging (MRI), the method comprising
electrically connecting a needle made according to claim 7 to electronic circuitry, wherein the at least one coil includes first and second coils;
generating sequences of MRI-pulses to acquire, with an MRI system, projection data representing three one-dimensional projections of the needle along three orthogonal spatial axes such as to determine a three-dimensional position of the at least one coil from sequenced projection data; and
generating data representing a position of the needle body by extrapolating the projection data along a direction connecting positions of the first and second coils.

9. A method according to claim 8, wherein the generating data representing a position of the needle body includes generating data representing a position of the needle tip.

10. A device configured for use with a system for actively tracking of a position of the device within a magnetic resonance imaging (MRI) scanner, the device comprising:
a needle including
a first metallic body portion having a first cylindrical surface, proximal and distal ends, a first length, and extended along an axis; and
a tubular element made of non-metallic and dia-magnetic material having a second length; and
at least one coil of an electrically-conductive member wound about the tubular element and electrically extended along the axis towards the proximal end,
wherein the distal end includes a second cylindrical surface having a radius that is smaller than a radius corresponding to the first cylindrical surface and the ratio of the first length to the second length is at least 10.

11. A device according to claim 10, wherein the needle further comprises a plastic sheath encasing at least the first metallic portion, wherein the first plastic portion and the plastic sheath are dimensioned to form a gap therebetween, and wherein an electrical extension of the coil is disposed in the gap.

12. A device according to claim 10, further comprising a programmable processor and a tangible non-transitory computer-readable medium containing computer program code thereon which, when the computer program is loaded on the processor and the system is operably cooperated with an MRI-system, causes the processor to
generate sequences of MRI-pulses to acquire four one-dimensional projections of the needle along three orthogonal spatial axes to determine three-dimensional positions of first and second coils.

13. A device according to claim 12, wherein the processor is programmed to create a visually-perceivable representation of a needle's tip, and wherein a projection of the four one-dimensional projections is used to create an image of the needle's tip that is devoid of artifacts cause by a motion of the needle within the scanner.

14. A device according to claim 12, wherein the processor is programmed to create a visually-perceivable representation of a needle's tip based on active tracking of the first and second coils.

15. A device according to claim 12, wherein the processor is further programmed to generate data representing a position of the needle by extrapolating the acquired data along a direction connecting positions of the first and second coils.

16. A device according to claim 12, wherein a sequence of MRI-pulses from said sequences includes multiple tracking segments of pulses, and wherein the processor is programmed to derive, from acquired projections of the needle and in response to a tracking segment of pulses, a corresponding data set representing position and orientation of the needle, said data set being derived in response to the sequence of MRI pulses that enables substantially artifact-free imaging of the needle in presence of a physiological motion of a body enclosing the needle.
